SEQUENCE LISTING (10 sequences)

SEQ ID NO: 1 - DNA, 1131 bp, Homo sapiens
atggcggactactggaagtcacagccaaagaaattctgtgattactgcaagtgctggatagcagacaataggcctagtgttgaatttcatgaaagaggaaagaatcataaggaaaatgtggcaaaaaggatcagtgagattaaacagaaaagcctggataaggcaaaggaagaagaaaaggcatcaaaggagtttgctgcaatggaggcagctgccctgaaagcataccaagaggatttgaaaagacttggcttagagtcagaaattttggagccaagcataacaccagtaaccagcactatcccacctacctcgacatcaaatcaacagaaagaaaagaaagaaaagaagaaaagaaaaaaagatccttcaaagggcagatgggtagaaggcataacctctgagggttaccattactattatgatcttatctcaggagcatctcagtgggagaaacctgaaggatttcaaggagacttaaaaaagacagcagtgaagaccgtttgggtagaaggtttaagtgaagatggttttacctattactataatacagaaacaggagaatccagatgggagaaacctgatgatttcattccacacactagtgatctgccttctagtaaggtcaatgaaaattcacttggcaccctagatgaatccaaatcatcagattcgcatagtgattctgatggggaacaggaagcagaagaaggaggggtctctacagagacagaaaagccaaaaataaagtttaaggaaaaaaataaaaatagtgatggaggaagtgacccagaaacacagaaagaaaaaagtattcagaaacagaattcattaggttcaaatgaagaaaaatcgaaaactcttaagaaatcaaacccatatggagaatggcaagaaattaaacaagaggttgagtctcatgaggaggtagatttggaacttccaagcactgaaaatgagtatgtatcaacttcagaagctgatggtggcggagaacccaaagtggtatttaaagaaaaaacagtcacttctcttggagttatggcagatggagtggccccagtcttcaaaaagagaagaactgaaaatggaaaatctagaaatttaaggcaacgaggtgatgatcaatag

SEQ ID NO: 2 - protein, 376 aa, Homo sapiens
MADYWKSQPKKFCDYCKCWIADNRPSVEFHERGKNHKENVAKRISEIKQKSLDKAKEEEKASKEFAAMEAAALKAYQEDLKRLGLESEILEPSITPVTSTIPPTSTSNQQKEKKEKKKRKKDPSKGRWVEGITSEGYHYYYDLISGASQWEKPEGFQGDLKKTAVKTVWVEGLSEDGFTYYYNTETGESRWEKPDDFIPHTSDLPSSKVNENSLGTLDESKSSDSHSDSDGEQEAEEGGVSTETEKPKIKFKEKNKNSDGGSDPETQKEKSIQKQNSLGSNEEKSKTLKKSNPYGEWQEIKQEVESHEEVDLELPSTENEYVSTSEADGGGEPKVVFKEKTVTSLGVMADGVAPVFKKRRTENGKSRNLRQRGDDQ

SEQ ID NO: 3 - DNA, 20 bp, Artificial Sequence
  Primer
ggagctgtcgtattccagtc

SEQ ID NO: 4 - DNA, 20 bp, Artificial Sequence
  Primer
aacccctcaagacccgttta

SEQ ID NO: 5 - DNA, 9342 bp, Artificial Sequence
  Plasmid
tcgactctagattgcggccgcggtcatagctgtttcctgaacatgtgatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaattccctatagtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcggcacgaggggagggaggttcgggtgggcatcgggcggctggaagagctcgactcgtcccgctgggaaagcgcgagtctgagtggaaccctggacgacttgcagagcggctggcgcagtcatggcggactactggaagtcacagccaaagaaattctgtgattactgcaagtgctggatagcagacaataggcctgtatgataattccgctgttagagattctaataataattgtgttgaatgaagtgctcctttttgatgtaaactcagtttacaaagcacttttatgtacattgctcttgcattttcaaaacatcagaagatttctgtcttctcagtcccctatgaaagctgtgagccttcttccttttcatcaacctgactttgattatatattaaattatttctctgtggccttttgggaaatgggcaatgagttggtcatatcattcatgttatgaagcacctatattataatggtggtgttcataatactgtcatacccaaagaattgtggaaagcccttaaaaaataaaagcaacatgtttagtttttcttgtatattttgtggtctagaaataatcaagtgtatttaatgcagagctgtgtatctttagatgcttaaatttgaaagaacaccattatttcctttctcatgaaacaaagtaacaactctgttaggtatgttgttagtctgtctcatagttgagatccagttttgaagatgtaaagcaaaaagcacaccgctgatttttctcccctcttgcatgtcaaaatactatgtacataccacataaacactagggtatctgttaaatatattttatgttgataccgtttttacttggcaaagttggatgcacaaagaatagtagtctcatattttaacaatgctgtggatttcttctgtagacatttatttcctgttacctgcaaggtgtttaggatttggatttttttttgatgttttggtctttttggatagttgctaaagttcttcactattttacctaggctatccttataaataaataccatgaatttttaaaaccacacaggtcacttaaagtctctctttcttggaggtacctaggctgtaaaatgaattttgtgagttatctagagtaagtaatcccttccagtgtagtctgtctttatcttcttgagttggtaatacacattgatttaacctttaattagaaacaaaattaatttaattttgaatggtcctttaatattgtattctttgtgtagtgtaatcaactttaggaaacgcaatgtatgaaaaacaggaggattatctgctttacaaagtcacactttaaagatgaattcatggtagtattttaaaatagctcagttaatgcatcatgtaagatttgattgacagaacttattttgcagagcttgtgaggaatttcttactgcatttaactagaaacttgtaatttagggagcatttctgattgtttaaatagataattgcgctcactcaccagcccctgtccccagtcatctagtggccatcactccccataggttatcatttttttttttcattttcttgcctatccttccattgtttctacatgcatgtagacacaagttcttattccagcatacataaaaggtaatgtactgtatatattattctgcactagttttaaaaaatttaacagtatgtaactggagctccctttgtatccatacataaagagcttctttctctctgttacagtagactatgagaatgtaccatagtttaaccagtcagtcccttagagatggataccgtattgttattgcaaacagtgctgcagtcagtaagcttgtgtatatcatgattttgtacatgtgcaggtatatctatagaataaattaccagaaatgaaattgctgtgtcaaagggtaaatgtatttgtagtcttgatagatattgccaaattatcctccgtagaagttgtacattttgaaccattaccacattgtataaaagtgtttgcttataatttacgaggaatcttaaaatacatacttttctcctaaatttaaggaataaagcaataaaatcattgttctctatgtccctccttgtgacaaatagagcaataaaattctaaaggcttttgatgttgaaagttgtttaagatttttttccccagtcttttggcttcatgtcacgtttgagtcctcattacaatcatagtcactagattagtcatcaagggtaccttgtatttttctccattaataaggtcatcatggtttattttaagaaccaaaggaaacctagtcagaggacataataataaacaatgtagttccctagcatcatttgattaaaagggccaatttgcattaaattaatgttttcatcttttctgccattcattttaattttaattttctcatgtttatgagaaaattatgtttactatgaattttatgatgtttatgtagttttcttttgttattttctcttttcattttcaagagtgttgaatttcatgaaagaggaaagaatcataaggaaaatgtggcaaaaaggatcagtgagattaaacagaaaagcctggataaggcaaaggaagaagaaaaggcatcaaaggagtttgctgcaatggaggcagctgccctgaaagcataccaagaggatttgaaaagacttggcttagagtcagaaattttggagccaagcataacaccagtaaccagcactatcccacctacctcgacatcaaatcaacagaaagaaaagaaagaaaagaagaaaagaaaaaaagatccttcaaagggcagatgggtagaaggcataacctctgagggttaccattactattatgatcttatctcaggagcatctcagtgggagaaacctgaaggatttcaaggagacttaaaaaagacagcagtgaagaccgtttgggtagaaggtttaagtgaagatggttttacctattactataatacagaaacaggagaatccagatgggagaaacctgatgatttcattccacacactagtgatctgccttctagtaaggtcaatgaaaattcacttggcaccctagatgaatccaaatcatcagattcgcatagtgattctgatggggaacaggaagcagaagaaggaggggtctctacagagacagaaaagccaaaaataaagtttaaggaaaaaaataaaaatagtgatggaggaagtgacccagaaacacagaaagaaaaaagtattcagaaacagaattcattaggttcaaatgaagaaaaatcgaaaactcttaagaaatcaaacccatatggagaatggcaagaaattaaacaagaggttgagtctcatgaggaggtagatttggaacttccaagcactgaaaatgagtatgtatcaacttcagaagctgatggtggcggagaacccaaagtggtatttaaagaaaaaacagtcacttctcttggagttatggcagatggagtggccccagtcttcaaaaagagaagaactgaaaatggaaaatctagaaatttaaggcaacgaggtgatgatcaatagttgcaggagagctttttgtacatgcttttaggacagaatggagacttatacacccaaagtttatctgtgtttgtttgtaagtattatgatgctaaaaatttagatttattctaaatgtatttgatgtgaattaaaataaatattttttcatgtgaaatttattttggttcctaaaatggaagcctaccacattgcattgtaatacagtgtattatgttcagtgtctaaaaactgctaattaagtcataatttaagatgctatgtatctgttatttaaaacatggagaaacagggcctttattccattcatattcataagagcatatttatcctgcattgaaaatgcattacttttgcacattgatattaactgttgtccaacaaataagtatcggagtacgtgagaatattcccagcccagtgatgatttggttctgaggctgatgtgagaaagctgatgtaaaaaatgtatgcatattggcttaccttactcaatttaaagtcaaaagccaacagccaggagcagctcaggtacttcagatgtgcttaatatggagtgaaaactggaccagaggtggaaagatgtatttgccccagttcacttcaaagcagcaaacgttgtttaagcattttagtttgaaccaggcattgtgttaggaatccagggataaagatgaatgaaatgtatttctgttcttcaggagttcacattctagcatatgccttttttcccccagtcatgcactctttttagcagtcttatatgtactagaaaagattttttaaagtttatttaaagctgtgtagatgaaggcttgatctaaaatagtataaggtctccagcttcagtaaaagtttttagtgtttacttaaattagtaatttctcactttctgtctggttaaaatgtcttaaaaagtagaattttcttctttaatccgtttagtagttacctcccttttacttttcaaattcacaaaattacatttctcataaattgtatagtatttaacttataatagttaatatttgtctttttaagatgactgtacatgtaagaaaaaagcttattaaaaacttgatcaaagaaaaaaaaaaaaaaaaaaaaaaa

SEQ ID NO: 6 - DNA, 7043 bp, Artificial Sequence
  Plasmid
attgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcggcacgaggggagggaggttcgggtgggcatcgggcggctggaagagctcgactcgtcccgctgggaaagcgcgagtctgagtggaaccctggacgacttgcagagcggctggcgcagtcatggcggactactggaagtcacagccaaagaaattctgtgattactgcaagtgctggatagcagacaatcggccgagtgttgaatttcatgaaagaggaaagaatcataaggaaaatgtggcaaaaaggatcagtgagattaaacagaaaagcctggataaggcaaaggaagaagaaaaggcatcaaaggagtttgctgcaatggaggcagctgccctgaaagcataccaagaggatttgaaaagacttggcttagagtcagaaattttggagccaagcataacaccagtaaccagcactatcccacctacctcgacatcaaatcaacagaaagaaaagaaagaaaagaagaaaagaaaaaaagatccttcaaagggcagatgggtagaaggcataacctctgagggttaccattactattatgatcttatctcaggagcatctcagtgggagaaacctgaaggatttcaaggagacttaaaaaagacagcagtgaagaccgtttgggtagaaggtttaagtgaagatggttttacctattactataatacagaaacaggagaatccagatgggagaaacctgatgatttcattccacacactagtgatctgccttctagtaaggtcaatgaaaattcacttggcaccctagatgaatccaaatcatcagattcgcatagtgattctgatggggaacaggaagcagaagaaggaggggtctctacagagacagaaaagccaaaaataaagtttaaggaaaaaaataaaaatagtgatggaggaagtgacccagaaacacagaaagaaaaaagtattcagaaacagaattcattaggttcaaatgaagaaaaatcgaaaactcttaagaaatcaaacccatatggagaatggcaagaaattaaacaagaggttgagtctcatgaggaggtagatttggaacttccaagcactgaaaatgagtatgtatcaacttcagaagctgatggtggcggagaacccaaagtggtatttaaagaaaaaacagtcacttctcttggagttatggcagatggagtggccccagtcttcaaaaagagaagaactgaaaatggaaaatctagaaatttaaggcaacgaggtgatgatcaatagttgcaggagagctttttgtacatgcttttaggacagaatggagacttatacacccaaagtttatctgtgtttgtttgtaagtattatgatgctaaaaatttagatttattctaaatgtatttgatgtgaattaaaataaatattttttcatgtgaaatttattttggttcctaaaatggaagcctaccacattgcattgtaatacagtgtattatgttcagtgtctaaaaactgctaattaagtcataatttaagatgctatgtatctgttatttaaaacatggagaaacagggcctttattccattcatattcataagagcatatttatcctgcattgaaaatgcattacttttgcacattgatattaactgttgtccaacaaataagtatcggagtacgtgagaatattcccagcccagtgatgatttggttctgaggctgatgtgagaaagctgatgtaaaaaatgtatgcatattggcttaccttactcaatttaaagtcaaaagccaacagccaggagcagctcaggtacttcagatgtgcttaatatggagtgaaaactggaccagaggtggaaagatgtatttgccccagttcacttcaaagcagcaaacgttgtttaagcattttagtttgaaccaggcattgtgttaggaatccagggataaagatgaatgaaatgtatttctgttcttcaggagttcacattctagcatatgccttttttcccccagtcatgcactctttttagcagtcttatatgtactagaaaagattttttaaagtttatttaaagctgtgtagatgaaggcttgatctaaaatagtataaggtctccagcttcagtaaaagtttttagtgtttacttaaattagtaatttctcactttctgtctggttaaaatgtcttaaaaagtagaattttcttctttaatccgtttagtagttacctcccttttacttttcaaattcacaaaattacatttctcataaattgtatagtatttaacttataatagttaatatttgtctttttaagatgactgtacatgtaagaaaaaagcttattaaaaacttgatcaaagaaaaaaaaaaaaaaaaaaaaaaatcgactctagattgcggccgcggtcatagctgtttcctgaacatgtgatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgaggaactgaaaaaccagaaagttaattccctatagtgagtcgtattaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatac

SEQ ID NO: 7 - DNA, 400 bp, Artificial Sequence
  Synthetic Sequence
atcaaatcaacagaaagaaaagaaagaaaagaagaaaagaaaaaaagatccttcaaagggcagatgggtagaaggcataacctctgagggttaccattactattatgatcttatctcaggagcatctcagtgggagaaacctgaaggatttcaaggagacttaaaaaagacagcagtgaagaccgtttgggtagaaggtttaagtgaagatggttttacctattactataatacagaaacaggagaatccagatgggagaaacctgatgatttcattccacacactagtgatctgccttctagtaaggtcaatgaaaattcacttggcaccctagatgaatccaaatcatcagattcgcatagtgattctgatggggaacaggaagcagaagaaggaggg

SEQ ID NO: 8 - DNA, 383 bp, Artificial Sequence
  Synthetic Sequence
tgtgatgctcggggatccgaattcaagcaagaaaagaaaaaaagatccttcaaagggcagatgggtagaaggcataacctctgagggttaccattactattatgatcttatctcaggagcatctcagtgggagaaacctgaaggatttcaaggagacttaaaaaagacagcagtgaagaccgtttgggtagaaggtttaagtgaagatggttttacctattactataatacagaaacaggagaatccagatgggagaaacctgatgatttcattccacgggatataagggaaaatgatatgaatgtgtatttttactcaaaataaaagaatcttctgaaaaaaaaaaaaaaaaaaaaaaaaaaaaaaaaaaaaagcttgcggc

SEQ ID NO: 9 - protein, 105 aa, Artificial Sequence
  Synthetic Sequence
KEKKEKKKRKKDPSKGRWVEGITSEGYHYYYDLISGASQWEKPEGFQGDLKKTAVKTVWVEGLSEDGFTYYYNTETGESRWEKPDDFIPHTSDLPSSKVNENSLG

SEQ ID NO: 10 - protein, 103 aa, Artificial Sequence
  Synthetic Sequence
DPNSSKKRKKDPSKGRWVEGITSEGYHYYYDLISGASQWEKPEGFQGDLKKTAVKTVWVEGLSEDGFTYYYNTETGESRWEKPDDFIPRDIRENDMNVYFYSK

(19) United States
(12) Patent Application Publication
(10) Pub. No.: US 2011/0027185 A1
(43) Pub. Date: Feb. 3, 2011

(54) Methods of Screening for Anti-Angiogenic Compounds

(76) Inventors: Barrie Wilkinson, Essex (GB); Christine Martin, Essex (GB); William Vousden, Essex (GB); Steven Moss, Essex (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/667,625

(22) PCT Filed: Jul. 14, 2008
(86) PCT No.: PCT/GB08/50563

§ 371 Date: Oct. 18, 2010

(30) Foreign Application Priority Data
Jul. 13, 2007 (GB) 0713563.5

(51) Int. Cl. A61K 39/395; G01N 33/566; C07K 16/18; A61K 49/00; A61K 31/365; C07D 313/00; A61P 35/00; A61P 29/00; A61P 17/06; A61P 27/02; A61P 9/10

(52) U.S. Cl. 424/9.2; 436/501; 435/7.21; 424/139.1; 530/387.9; 514/450; 549/271

(57) ABSTRACT

The present invention relates to methods of screening for agents that bind to FBP21. In particular the present invention provides a method of identifying anti-angiogenic agents by screening for agents which are able to bind to FBP21. Compounds identified using the methods and screens of the present invention are useful in the prevention and treatment of conditions associated with angiogenesis.

The present invention relates to methods of screening for agents that bind to FBP21. In particular the present invention provides a method of identifying anti-angiogenic agents by screening for agents which are able to bind to FBP21. Compounds identified using the methods and screens of the present invention are useful in the prevention and treatment of conditions associated with angiogenesis.

### BACKGROUND OF THE INVENTION

Borrelidin 1 (FIG. 1) is an 18-membered macrolide produced by several bacterial strains including, but not limited to, Streptomyces rochei ATCC23956, Streptomyces parvulus Tü113 and Streptomyces parvulus Tü4055.

Several groups have reported the synthesis of fragments of the borrelidin structure, and four independent total syntheses of borrelidin have been reported (Hanessian et al., 2003; Duffey et al., 2003; Nagamitsu et al., 2004; Vong et al., 2004). In addition the gene cluster responsible for the biosynthesis of borrelidin by Streptomyces parvulus Tü4055 has been identified, cloned and sequenced (WO 2004/058976; Olano et al., 2004a). Based on this the application of biosynthetic engineering techniques have allowed elucidation of the biosynthetic pathway leading to borrelidin and to the production of new analogues (WO 2004/058976; Olano et al., 2003; Olano et al., 2004b; Moss et al., 2006).

Borrelidin was first discovered due to its antibacterial activity (Berger et al., 1949), although this antibacterial activity extends only to a limited number of micrococci, and is not found against all common test bacteria. The mode of action in sensitive microorganisms involves selective inhibition of threonyl tRNA synthetase (Paetz & Nass, 1973; Ruan et al., 2005). Other activities against spirochetes of the genus Treponema (Singh et al., 1985; U.S. Pat. No. 4,759,928), against viruses (Dickinson et al., 1965), uses for the control of animal pests and weeds (DE 3607287) and use as an agricultural fungicide (DE 19835669; U.S. Pat. No. 6,193,964) have been reported. Additionally, recently, borrelidin has been reported to have antimalarial activity against drug resistant Plasmodium falciparum strains (Otoguro et al., 2003).

A disclosure of particular interest is the discovery that borrelidin displays anti-angiogenesis activity (Wakabayashi et al., 1997). Angiogenesis is the process of the formation of new blood vessels. Angiogenesis occurs only locally and transiently in adults, being involved in, for example, repair following local trauma and the female reproductive cycle. It has been established as a key component in several pathogenic processes including cancer, rheumatoid arthritis and diabetic retinopathy (Perrin et al., 2005). Its importance in enabling tumours to grow beyond a diameter of 1-2 mm was established by Folkman (Folkman, 1986, Carmeliet, 2005; Ferrara and Kerbel, 2005), and is provoked by the tumour responding to hypoxia. In its downstream consequences angiogenesis is mostly a host-derived process, thus inhibition of angiogenesis offers significant potential in the treatment of cancers, avoiding the hurdles of other anticancer therapeutic modalities such as the diversity of cancer types and drug resistance (Matter, 2001).

In the rat aorta matrix culture model of angiogenesis, borrelidin exhibits a potent angiogenesis-inhibiting effect and also causes disruption of formed capillary tubes in a dose dependent manner by inducing apoptosis of the capillary-forming cells (Wakabayashi et al., 1997). Borrelidin inhibited capillary tube formation with an IC₅₀ value of 0.4 ng/mL (0.8 nM). In the same study, borrelidin was shown to possess anti-proliferative activity towards human umbilical vein endothelial cells (HUVEC) in a cell growth assay; the IC₅₀ value was measured at 6 ng/ml, which is 15-fold weaker than the anti-angiogenesis activity measured in the same medium. This anti-proliferative activity of borrelidin was shown to be general towards various cell lines. In addition to these data the authors report that borrelidin inhibits tRNA synthetase and protein synthesis in the cultured rat cells; however the IC₅₀ value for anti-angiogenesis activity (0.4 ng/ml) was 50-fold lower than that reported for inhibition of protein synthesis (20 ng/ml), indicating different activities of the compound.

Borrelidin also displays potent inhibition of angiogenesis in vivo using the mouse dorsal air sac model (Funahashi et al., 1999), which examines VEGF-induced angiogenesis and is an excellent model for studying tumour-angiogenesis. Borrelidin was administered at a dose of 1.8 mg/kg by intraperitoneal injection and shown to significantly reduce the increment of vascular volume induced by WiDr cells, and to a higher degree than does TNP-470, which is a synthetic angiogenesis inhibitor in clinical trials. Detailed controls verified that these data are for angiogenesis inhibition and not inhibition of growth of the tumour cells. The authors also showed that borrelidin is effective for the inhibition of the formation of spontaneous lung metastases of B16-BL6 melanoma cells at the same dosage by inhibiting the angiogenic processes involved in their formation.

JP 9-227,549 and JP 8-173,167 confirm that borrelidin is effective against WiDr cell lines of human colon cancer, and also against PC-3 cell lines of human prostate cancer. JP 9-227,549 describes the production of borrelidin by Streptomyces rochei Mer-N7167 (Ferm P-14670) and its isolation from the resulting fermentation culture.

WO 01/09113 discloses the preparation of borrelidin analogues that have undergone synthetic modification at the carboxylic acid moiety of the cyclopentane ring. The activity of these compounds was examined using endothelial cell proliferation and endothelial capillary formation assays in a similar manner to that described above. In general, modification of the carboxyl moiety improved the selectivity for inhibiting capillary formation: the major reason for this improvement in selectivity is through a decrease in the cell proliferation inhibition activity whereas the capillary formation inhibitory activity was altered to a much lower degree.

Specifically, the borrelidin-morpholinoethyl ester showed a 60-fold selectivity index, the borrelidin-amide showed a 37-fold selectivity index, the borrelidin-(2-pyridyl)-ethyl ester showed a 7.5-fold selectivity index and the borrelidin-morpholinoethyl amide showed a 6-fold selectivity index, for the capillary formation inhibitory activity versus cell proliferation with respect to borrelidin. The capillary formation inhibitory activity of these and other borrelidin derivatives was verified using a micro-vessel formation assay. In addition, the authors showed that borrelidin weakly inhibited the propagation of metastatic nodules, after removal of the primary tumour, when using a Lewis lung adenocarcinoma model. However, the borrelidin-(3-picolylamide) derivative was reported to inhibit very considerably the increase of micrometastases in rats after intraperitoneal and also with per os administration at subtoxic doses. Similarly, using the colon 38 spleen liver model, the metastasis-forming ability of mouse colon adenocarcinoma cells transplanted into mouse spleen was considerably decreased after treatment with a subtoxic dose of this borrelidin derivative. These data confirm the earlier reported ability of borrelidin and its derivatives to inhibit the formation of metastases.

Two further reports have been published concerning the biological activity of borrelidin. The first of these indicates that the anti-angiogenic effects of borrelidin are mediated through distinct pathways (Kawamura et al., 2003). High concentrations of threonine were found to attenuate the ability of borrelidin to inhibit both capillary tube formation in the rat aorta culture model and HUVEC cells proliferation; however, it did not affect the ability of borrelidin to collapse formed capillary tubes or to induce apoptosis in HUVEC. Borrelidin was also found to activate caspase-3 and caspase-8, and inhibitors of both of these suppressed borrelidin induced apoptosis in HUVEC. The second of these papers used the method of global cellular mRNA profiling to provide insight into the effects of borrelidin on Saccharomyces cerevisiae (Eastwood and Schaus, 2003). This analysis showed the induction of amino acid biosynthetic enzymes in a time-dependent fashion upon treatment with borrelidin, and it was ascertained that the induction of this pathway involves the GCN4 transcription factor.

Angiogenesis is crucial to tumour growth and metastasis and the development of solid tumors beyond 1-2 mm in diameter has been established to require angiogenesis. It is a multifaceted process and involves the coordination of proliferation, migration, adhesion and tubule formation of the endothelial cells. The therapeutic utilities of angiogenesis inhibitors in cancer treatment have been demonstrated by the clinical efficacies of bevacizumab and thalidomide for example. Anti-angiogenic activity is also an important component of some widely used chemotherapeutics such as paclitaxel, and the most recently marketed target-based therapeutics such as sunitinib and sorafenib. Therefore, angiogenesis inhibition is a validated approach to the development of anticancer therapeutics.

The angiogenesis-inhibitory effect of borrelidin is directed towards the twin biological effects of proliferation and capillary formation (Wilkinson et al., 2006). In addition, borrelidin, and derivatives thereof, have been shown to inhibit the propagation of metastases. Borrelidin also has indications for use in cell cycle modulation. Thus, borrelidin and related compounds are particularly attractive targets for investigation as therapeutic agents for the treatment of tumour tissues, either as single agents or for use as an adjunct to other therapies. In addition, they may be used for treating other diseases in which angiogenesis is implicated in the pathogenic process, including, but not restricted to, the following list: rheumatoid arthritis, psoriasis, atherosclerosis, diabetic retinopathy and various ophthalmic disorders. Thus borrelidin is an useful lead molecule for use in the treatment of these various diseases in which angiogenesis is responsible for their pathogenesis, and the identification of the mode of action is of significant value and fundamental importance.

It is of interest therefore to determine the molecular target to which borrelidin directly binds, and by which it exerts its angiogenesis inhibitory activity. Many methods are available for identifying the cellular molecular targets of molecules, including, but not limited to, phage display bio-panning (McKenzie et al., 2004; Jin et al., 2002), affinity chromatography (Yamaoka et al., 2005; Usui et al., 2005) and yeast 2-hybrid/ yeast 3-hybrid screening (Caligiuri, M., 2005).

In order to identify the molecular target of borrelidin we chose to utilise phage display biopanning. Several examples of this approach to identify the molecular target of natural products have been reported (McKenzie et al., 2004; Jin et al., 2002). Using the phage display biopanning methods described in more detail herein forming binding protein 21 (FBP21) was identified by the Inventors as a target of borrelidin, and apparently interacts with one or both of the WW domains thereof. The direct biological function of FBP21 is unknown but it has been implicated to have a role in the formation and function of the spliceosome (Bedford et al., 1998). The fact that the WW domains region of this molecule was identified in our experiments indicates that this ought to be the site of action of borrelidin and that borrelidin may affect the function of FBP21 by blocking its ability to mediate protein-protein interactions between itself and other proteins, potentially those involved in the formation and/or function of the spliceosome. FBP21 is known to contain 2 WW domains adjacent to one another.

Many genes contain several introns and alternative splicing enables a single gene to increase its coding capacity allowing the synthesis of structurally and functionally distinct protein isoforms. The generation of alternative isoforms of an individual gene is controlled by the spliceosome. The spliceosome is a macromolecular machine responsible for the maturation of pre-mRNA's to mature mRNA through the specific removal of introns from pre-mRNA (Sanford et al., 2004). The spliceosome consists of many components coming together, in part, around the C-terminal domain (CTD) of the RNA polymerase II (Pol II) large subunit. Of particular importance are the five, U-rich, small ribonucleoproteins (snRNPs) U1, U2, U4, U5, U6, and the serine-arginine (SR) rich proteins. Many other proteins have been identified to be associated with the spliceosome for which no function has been ascribed (Sanford et al., 2004).

The precise manner in which alternative splicing is regulated in unknown, but it is clear that the nature of intron and exon coding sequences, the rate of function (processivity) of Pol II, as well as the recruitment of different splicing factors and related components play a role. Mutation is also a factor and indeed 15% of mutations that cause genetic disease affect pre-mRNA splicing (Krawczak et al., 1992).

The finding that FBP21 is a target of borrelidin has ramifications for the potential mechanism of action of borrelidin, as evidence indicates that FBP21 (and several related WW domain containing proteins such as FBP11 (Lin et al., 2004)) is involved in spliceosomal assemblage (Bedford et al., 1998). Specifically, FBP21 has been identified to be present in a highly purified sample of the spliceosomal complex A, as associating with U2 snRNPs, and as co-localising with splicing factors in nuclear speckle domains (Bedford et al., 1998). Moreover, FBP21 interacts directly with the U1 snRNP U1C, the core snRNP proteins SmB and SmB′, and the branchpoint binding protein SF1/mBBP domains (Bedford et al., 1998). This indicates that FBP21 plays an important role spliceosome assembly and/or function.

The importance of alternative splicing in the process of angiogenesis and the pathology of certain disease states has been identified (Bates et al, 2002; Bates et al, 2004; Perrin et al, 2005) and other antitumor agents have been identified that affect other parts of the spliceosome, such as pladienolide and spliceostatin, which have been found to affect the the splicing factor SF3b (Kotake et al., 2007, Kaida et al., 2007).

WW domains are protein motifs involved in protein-protein interactions and often have effects ion signalling pathways (Chan and Leder, 1996, Chan et al., 1996).

Tumour growth, survival and metastasis are underpinned by the creation of new blood vessels. The physiological or pathological formation of new vessels is a complex process controlled by at least 10-endothelium cell specific growth factors from the vascular endothelial cell growth factors (VEGF), angiopoieten and ephrin families (Ferra and Davis-Smyth, 1997). Other non-endothelial cell-specific growth factors, molecules and enzymes also play a role.

Although the exact manner by which tumour vascularisation is controlled is still not fully understood, VEGF-A appears to be predominant in most tumours and its inhibition has great potential in relation to, and has attracted significant attention for, anti-angiogenesis based cancer therapies.

Exon slicing of the VEGF pre-mRNA results in three main mRNAs that encode for three secreted isoforms, VEGF₁₈₉, VEGF₁₆₅, and VEGF₁₂₁; a number of minor isoforms also exist (Ferra and Davis-Smyth, 1997). The numbers associated with the isoforms indicate the number of amino acid residues in its length, i.e. 165 residues for VEGF₁₆₅. An increase in VEGF mRNA expression has been identified in almost all known tumours (Ferra and Davis-Smyth, 1997).

In addition to these VEGF splice variants, a second series of related splice variants have been identified (Bates et al., 2002). These are of the same size as those described above but vary due to alternative splicing from the end of exon 7 into the untranslated region of the mRNA. Cloning of the alternative transcript for VEGF₁₆₅ showed that translation would result in a 165-amino acid peptide with an alternative terminal 6 amino acids. This isoform was termed VEGF₁₆₅b. Similar alternatively spliced ‘b’ isoforms exist for the other VEGF splice variants.

These and subsequent experiments showed that the ‘b’ series of alternatively spliced isoforms were anti-angiogenenic in contrast to the normal isoforms which are strongly pro-angiogenic (Bates et al., 2002, Woolard et al., 2004). Moreover, the ‘b’ isoform VEGF₁₆₅b is down regulated in renal (Bates et al., 2002) and prostate tumours (Woolard et al., 2004).

Further studies showed that VEGF₁₆₅b is expressed in normal tissues and is present in the circulation (Woolard et al., 2004). VEGF₁₆₅b binds with equal affinity to the VEGF receptor 2 but does not activate it or stimulate downstream signalling pathways. Moreover, it prevents VEGF receptor 2 phosphorylation and signalling in cultured cells. VEGF₁₆₅b was also shown, using two different in vivo models that it inhibits VEGF₁₆₅ mediated angiogenesis in rabbit cornea and rat mesentery. VEGF₁₆₅b expressing tumours were shown to grow significantly more slowly than VEGF₁₆₅ expressing tumours indicating that a switch in splicing from VEGF₁₆₅ to VEGF₁₆₅b can inhibit tumour growth. These results indicate that regulation of VEGF alternative splicing may be a critical switch from an anti-angiogenic to a pro-angiogenic phenotype.

Diabetic retinopathy is a disease in which angiogensis plays a significant role in pathology. It has been shown that in the eyes of diabetic retinopathy patients VEGF splicing was switched from an anti-angiogenic to a pro-angiogenic environment (Perrin et al., 2005). This occurred through changes to the ratio of VEGFₓₓₓ to VEGF_{xxxb}, e.g., VEGF₁₆₅ to VEGF₁₆₅b. Alterations to splicing and through that to the balance of VEGF isoforms could therefore be of potential therapeutic value.

Small molecule inhibitors of this switch in alternative splicing for the VEGF isoforms may represent useful therapeutic therapies.

The present inventors have identified that FBP21 is a target of borrelidin, a compound known to have potent antiangiogenic activities, therefore, the present invention provides methods for screening for compounds which are able to bind to FBP21, in particular the WW domains of FBP21. These compounds have utility in the treatment of cancer and B-cell malignancies and other disorders in which angiogenesis is implicated in the pathogenic process.

### SUMMARY OF THE INVENTION

The present invention provides a method of identifying a compound that interacts with (eg binds to) a FBP21 polypeptide such as FBP21. In particular the present invention provides a method for identifying compounds that interact with (eg bind to) one or both of the WW domains of a FBP21 polypeptide (eg FBP21).

The present invention also provides a method for screening for anti-angiogenic agents which comprises identifying agents that interact with (eg bind to) a FBP21 polypeptide e.g. FBP21 or a FBP21 polypeptide fragment.

For example the agents interact with one or both of the WW domains of the FBP21 polypeptide (eg FBP21) or FBP21 polypeptide fragment.

The present invention also provides kits suitable for use in the above methods.

In a further aspect the present invention provides a FBP21 polypeptide (eg FBP21) or a FBP21 polypeptide fragment attached to a solid support for use in a method of the invention.

In another aspect the present invention provides for the use of a compound e.g. identified via the methods disclosed herein which interacts with (eg binds to) FBP21 thereby to disrupt the interaction of FBP21 with its natural ligand for use in the manufacture of a medicament for use in treating conditions associated with angiogenesis such as tumours. It also provides an anti-angiogenic agent which interacts with (eg binds to) FBP21 thereby to disrupt the interaction of FBP21 with its natural ligand for use in the treatment of conditions associated with angiogenesis such as tumours. It also provides a method of treatment of conditions associated with angiogenesis which comprises administering to a patient in need thereof an anti-angiogenic agent which binds to FBP21 thereby to disrupt the interaction of FBP21 with its natural ligand. In such methods and uses the agent may suitably bind to one or both of the WW domains of FBP21.

In one embodiment of the invention it is of interest that agents interact with one WW domain of FBP21 or a FBP21 polypeptide or a FBP21 polypeptide fragment. In another embodiment of the invention it is of interest that agents interact with two WW domains of FBP21 or a FBP21 polypeptide or a FBP21 polypeptide fragment.

### DEFINITIONS

As used herein the term “analogue(s)” refers to chemical compounds that are structurally similar to another but which differ slightly in composition (as in the replacement of one atom by another or in the presence or absence of a particular functional group).

As used herein, the term “homologue(s)” refers a homologue of a gene or of a protein encoded by a gene disclosed herein. Such homologue(s) encode a protein that performs the same function or can itself perform the same function as said gene or protein. Preferably, such homologue(s) have at least 40% sequence identity, preferably at least 60%, at least 70%, at least 80%, at least 90% or at least 95% sequence identity to the sequence of the particular gene or protein disclosed herein (taking the whole gene sequence or protein sequence as the comparator). Percentage identity may be calculated using any program known to a person of skill in the art such as BLASTn or BLASTp, available on the NCBI website.

As used herein, the term “FBP21 polypeptide” refers to a polypeptide which:
a) comprises an amino acid sequence according to SEQ ID NO: 2; or
b) is a derivative of a) having one or more amino acid substitutions, modifications, deletions or insertions, and which retains the activity of the FBP21 polypeptide. As used herein, the term “FBP21 polypeptide fragment” refers to a polypeptide which is:
c) a fragment of an FBP21 polypeptide according to a) or b) above, which is at least 23 amino acids long (eg at least 80 amino acids long, for example at least 100 amino acids long) and encodes one or more WW domains and has at least 70% sequence identity (eg at least 80% suitably at least 90% e.g. at least 95% sequence identity) to SEQ ID NO: 2 over the length of the fragment.

The term “polypeptides” includes peptides, polypeptides and proteins. These are used interchangeably unless otherwise specified

As used herein, the term “FBP21 nucleic acid” refers to a nucleic acid which
d) comprises a DNA sequence which codes for a FBP21 polypeptide as defined in a) or a FBP21 polypeptide fragment as defined in c), or the RNA equivalent of either;
e) has a sequence which is complementary to the sequences of d);
f) has a sequence which codes for a polypeptide as defined in a) to c) above; or
g) has a sequence which shows substantial identity with any of those of d), e) and f);

Unless the context indicates otherwise, FBP21 nucleic acids include those nucleic acid molecules defined in d) to g) above and may have one or more of the following characteristics:
1) they may be DNA or RNA;
2) they may be single or double stranded;
3) they may be in substantially pure form. Thus, they may be provided in a form which is substantially free from contaminating proteins and/or from other nucleic acids; and
4) they may be with introns or without introns (e.g. as cDNA).

An FBP21 nucleic acid according to g) above which encodes an FBP21 polypeptide may show greater than about 60% identity with the sequence of SEQ ID NO: 1 (taking the sequence of SEQ ID NO: 1 as the comparison window), greater than about 70% identity, greater than about 80% identity, or greater than about 90%, 91%, 92%, 93%, 94%, 95%, 96%, 97%, 98% or 99% identity therewith. Percentage identity may be calculated using one of the programs such as BLAST or BestFit from within the Genetics Computer Group (GCG) Version 10 software package available from the University of Wisconsin, using default parameters.

In preferred embodiments, whether coding or non-coding, nucleotide sequences suitable for use in the present invention are capable of hybridising specifically with at least a portion of the sequence of SEQ ID NO: 1 or the complement thereof.

An FBP21 polypeptide according to b) above may show greater than about 60% identity with the sequence of SEQ ID NO: 2 (taking the sequence of SEQ ID NO: 2 as the comparison window), greater than about 70% identity, greater than about 80% identity, or greater than about 90%, 91%, 92%, 93%, 94%, 95%, 96%, 97%, 98% or 99% identity therewith. Percentage identity may be calculated using one of the programs such as BLAST or BestFit from within the Genetics Computer Group (GCG) Version 10 software package available from the University of Wisconsin, using default parameters.

For example, hybridizations may be performed, according to the method of Sambrook et al. (Sambrook et al., 1989), using a hybridization solution comprising: 5×SSC, 5× Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C.+16.6Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such hybridisation would be considered substantially specific to the nucleic acid sequence of the present invention.

The nucleic acids of the present invention preferably comprise at least 15 contiguous nucleotides of SEQ ID NO: 1. They may comprise 20, 25, 30, 35, 40, 45, 50, 60, 70, 80, 90, 100, 120, 150, 200, 300, 500 or more contiguous nucleotides of SEQ ID NO: 1.

As used herein, the term “diseases associated with angiogenesis” or “conditions associated with angiogenesis” refers to, without limitation, cancer and anti-B-cell malignancy, rheumatoid arthritis, psoriasis, atherosclerosis, diabetic retinopathy, macular degeneration and various ophthalmic disorders. Diseases/conditions associated with angiogenesis include tumours.

### DETAILED DESCRIPTION OF THE INVENTION

Phage display technology was used to screen a peptide phage display library to identify peptides that bind to borrelidin or an active analogue thereof. Methods for preparing libraries containing diverse populations of various types of molecules such as peptides, polypeptides, proteins, and fragments thereof are known in the art. Phage display libraries are also commercially available.

To enable this method of screening it was first necessary to chemically modify borrelidin in such a manner as not to affect its biological activity. This was accomplished by attaching a biotin ligand via a flexible linker (spacer group). To do this borrelidin and pre-borrelidin were activated by reaction with isobutyl chloroformate and the coupled with biotinamidohexanoic acid hydrazine to generate the biotinylated species separated by a C6 spacer group (Example 1, FIG. 2).

In addition, the use of a negative control in each assay can be of value in order to eliminate non-specific background hits unrelated to the activity of the compound. Such an inactive control will ideally be an analogue of borrelidin carrying a simple, preferably single, chemical modification which inactivates the molecule but does not cause significant perturbation to the three-dimensional structure of the ligand. In the case of borrelidin, a useful negative control is pre-borrelidin which shares much of the structure of the compound but which lacks any activity (see Example 2).

Selection of the appropriate phage display library is routine to a person of skill in the art. Ideally the library should be one related to the pathology of disease or mode of action of the molecule, i.e. for borrelidin a suitable selection includes a library derived from a cancer tumour cell line or from an endothelial cell such as human umbilical cord endothelial cells (HUVEC).

Essentially, a library of phage displaying potential binding peptides was incubated with biotinylated borrelidin to select clones encoding recombinant peptides that specifically bind the borrelidin. After at least one round of biopanning (binding to the biotinylated borrelidin), the phage DNA was amplified and sequenced, thereby providing the sequence for the displayed binding peptides. The binding phage can then be collected and amplified following elution. Secondary and tertiary pannings can be performed as necessary. Following the last screening, individual colonies of phage-infected bacteria can be picked at random, the phage DNA isolated and subjected to automated DNA sequencing. The sequence of the displayed peptides was deduced from the DNA sequence and FBP21 (specifically via its WW domains) was identified as a protein which bound to borrelidin but not to pre-borrelidin, an inactive analogue of borrelidin (see Examples).

Therefore, the present inventors have identified that FBP21 polypeptides represent a suitable target for the treatment of diseases associated with angiogenesis. Thus, in one aspect, the present invention provides methods for identifying agents that are capable of interacting with or modulating the expression or activity of an FBP21 polypeptide or the expression of an FBP21 nucleic acid molecule. Agents identified through the screening methods of the invention are potential therapeutics for use in the treatment of diseases associated with angiogenesis. The present invention relates to methods of screening for agents that bind to FBP21. These agents are useful in preventing or inhibiting angiogenesis.

It is among the objects of the present invention to provide a method of screening for agents which bind to FBP21 and which thereby modulate the activity of FBP21 in the control of alternative splicing. In a specific embodiment the present invention provides a method of screening for agents which bind to one or both of the WW domains of FBP21.

Therefore, in one aspect the present invention provides methods of screening for agents that interact with an FBP21 polypeptide or a FPB21 polypeptide fragment, said method comprising:

(i) contacting said polypeptide or polypeptide fragment with a candidate agent; and

(ii) determining whether or not the candidate agent interacts with said polypeptide or polypeptide fragment.

In the following text, references to “FPB21 polypeptide” embrace references to “FBP21 polypeptide fragment” unless the context demands otherwise.

Preferably, the determination of an interaction between the candidate agent and the FBP21 polypeptide comprises quantitatively detecting binding of the candidate agent and said polypeptide.

More preferably the method further comprises selecting an agent, which interacts with an FBP21 polypeptide or is capable of modulating the interaction, expression or activity of an FBP21 polypeptide, for further testing for use in the treatment and/or prophylaxis of diseases associated with angiogenesis. It will be apparent to one skilled in the art that the above screening methods are also appropriate for screening for agents which interact with or modulate the expression or activity of an FBP21 nucleic acid.

The invention also provides assays for use in drug discovery in order to identify or verify the efficacy of agents for treatment or prophylaxis of diseases associated with angiogenesis. Agents identified using these methods can be used as lead agents for drug discovery, or used therapeutically.

Expression of an FBP21 polypeptide can be assayed by, for example, immunoassays, gel electrophoresis followed by visualisation, detection of mRNA or FBP21 polypeptide activity, or any other method taught herein or known to those skilled in the art. Such assays can be used to screen candidate agents, in clinical monitoring or in drug development.

Agents can be selected from a wide variety of candidate agents. Examples of candidate agents include but are not limited to, nucleic acids (e.g. DNA and RNA), carbohydrates, lipids, proteins, polypeptides, peptides, peptidomimetics, small molecules, antibodies, polyketides and other drugs. In particular, agents may be selected from polyketides. Agents can be obtained using any of the numerous approaches in combinatorial library methods known in the art, including: biological libraries, spatially addressable parallel solid phase or solution phase libraries; synthetic library methods requiring deconvolution; the “one-bead one-compound” library method; and synthetic library methods using affinity chromatography selection. The biological library approach is suited to peptide libraries, while the other four approaches are applicable to peptide, non-peptide oligomer or small molecule libraries of compounds (Lam, 1997; U.S. Pat. No. 5,738,996; and U.S. Pat. No. 5,807,683).

In one embodiment the agent is a small molecule (eg having a molecular weight of less than 1000 e.g. less than 500 e.g. less than 400 Da).

In another embodiment the agent is an antibody e.g. a monoclonal antibody (e.g. a human or humanised monoclonal antibody).

Examples of suitable methods based on the present description for the synthesis of molecular libraries can be found in the art, for example in: DeWitt et al., 1993,; Erb et al., 1994, Zuckermann et al., 1994, Cho et al., 1993, Carrell et al., 1994, Carell et al., 1994, and Gallop et al., 1994,.

Libraries of compounds may be presented, for example, in solution (e.g. Houghten, 1992), or on beads (Lam, 1991), on chips (Fodor, 1993), bacteria (US 5,223, 409), spores (U.S. Pat. Nos. 5,571,698; 5,403,484; and 5,223,409), plasmids (Cull et al., 1992) or phage (Scott and Smith, 1990, Devlin, 1990, Cwirla et al., 1990, Felici, 1991).

A FBP21 polypeptide (eg FBP21) may for example be produced by cells in situ. Thus in one embodiment, agents that interact with (e.g. bind to) an FBP21 polypeptide are identified in a cell-based assay where a population of cells expressing an FBP21 polypeptide is contacted with a candidate agent and the ability of the candidate agent to interact with the polypeptide is determined. Preferably, the ability of a candidate agent to interact with an FBP21 polypeptide is compared to a reference range or control. In another embodiment, a first and second population of cells expressing an FBP21 polypeptide are contacted with a candidate agent or a control agent and the ability of the candidate agent to interact with the polypeptide is determined by comparing the difference in interaction between the candidate agent and control agent, for example a suitable control agent is an agent known to interact with FBP21, e.g. borrelidin. If desired, this type of assay may be used to screen a plurality (e.g. a library) of candidate agents using a plurality of cell populations expressing an FBP21 polypeptide. If desired, this assay may be used to screen a plurality (e.g. a library) of candidate agents.

The cell can be, for example, of prokaryotic origin (e.g. E. coli) or eukaryotic origin (e.g. yeast or mammalian). Further, the cells can express the FBP21 polypeptide endogenously or be genetically engineered to express the polypeptide. In some embodiments, an FBP21 polypeptide or the candidate agent is labelled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde orfluorescamine) to enable detection of an interaction between a polypeptide and a candidate agent.

In another embodiment, agents that interact with (e.g. bind to) an FBP21 polypeptide are identified in a cell-free assay system where a sample expressing an FBP21 polypeptide is contacted with a candidate agent and the ability of the candidate agent to interact with the polypeptide is determined. Preferably, the ability of a candidate agent to interact with an FBP21 polypeptide is compared to a reference range or control. In a preferred embodiment, a first and second sample comprising native or recombinant FBP21 polypeptide are contacted with a candidate agent or a control agent and the ability of the candidate agent to interact with the polypeptide is determined by comparing the difference in interaction between the candidate agent and control agent. If desired, this assay may be used to screen a plurality (e.g. a library) of candidate agents using a plurality of FBP21 polypeptide samples.

Preferably, the polypeptide is first immobilized, by, for example, contacting the polypeptide with an immobilized antibody which specifically recognizes and binds it, or by contacting a purified preparation of polypeptide with a surface designed to bind proteins. The polypeptide may be partially or completely purified (e.g. partially or completely free of other polypeptides) or part of a cell lysate. Further, the polypeptide may be a fusion protein comprising the PTK7 polypeptide or a biologically active portion thereof and a domain such as glutathionine-S-transferase. Alternatively, the polypeptide can be biotinylated using techniques well known to those of skill in the art (see Examples, other methods are described in e.g. biotinylation kit, Pierce Chemicals; Rockford, Rigaut et al., 1999; Husi,. 2000, Ho, et al., 2002, Gavin,.et al., 2002).

In one embodiment, an FBP21 polypeptide is used as a “bait protein” in a two-hybrid assay or three hybrid assay to identify other proteins that bind to or interact with the FBP21 polypeptide (see e.g. U.S. Pat. No. 5,283,317; Zervos et al., 1993, Madura et al. 1993, Bartel et al., 1993,; Iwabuchi et al., 1993,; and WO 94/10300). As those skilled in the art will appreciate, such binding proteins are also likely to be involved in the same pathways and mechanisms as the FBP21 polypeptide. For example, they may be upstream or downstream elements of a signalling pathway involving an FBP21 polypeptide or they may also be involved in the regulation of alternative splicing. Alternatively, polypeptides that interact with an FBP21 polypeptide can be identified by isolating a protein complex comprising an FBP21 polypeptide (i.e. an FBP21 polypeptide which interacts directly or indirectly with one or more other polypeptides) and identifying the associated proteins using methods known in the art such as mass spectrometry or Western blotting (for examples see Blackstock, & Weir, 1999, Rigaut, G. 1999,; Husi, 2000, Ho, Y. et al., 2002, Gavin, A. et al., 2002).

In all cases, the ability of the candidate agent to interact directly or indirectly with the FBP21 polypeptide can be determined by methods known to those of skill in the art. For example but without limitation, the interaction between a candidate agent and an FBP21 polypeptide can be determined by flow cytometry, a scintillation assay, an activity assay, mass spectrometry, microscopy, immunoprecipitation or western blot analysis.

In yet another embodiment, agents that competitively interact with (i.e. competitively binding to) an FBP21 polypeptide are identified in a competitive binding assay and the ability of the candidate agent to interact with the FBP21 polypeptide is determined. Preferably, the ability of a candidate agent to interact with an FBP21 polypeptide is compared to a reference range or control. In a preferred embodiment, a first and second population of cells expressing both an FBP21 polypeptide and a protein or other agent which is known to interact with the FBP21 polypeptide are contacted with a candidate agent or a control agent. The ability of the candidate agent to competitively interact with the FBP21 polypeptide is then determined by comparing the interaction in the first and second population of cells. In another embodiment, an alternative second population or a further population of cells may be contacted with an agent which is known to competitively interact with an FBP21 polypeptide. Alternatively, agents that competitively interact with an FBP21 polypeptide are identified in a cell-free assay system by contacting a first and second sample comprising an FBP21 polypeptide and a protein known to interact with the FBP21 polypeptide with a candidate agent or a control agent. The ability of the candidate agent to competitively interact with the FBP21 polypeptide is then determined by comparing the interaction in the first and second sample. In another embodiment, an alternative second sample or a further sample comprising an FBP21 polypeptide may be contacted with an agent which is known to competitively interact with an FBP21 polypeptide. In any case, the FBP21 polypeptide and known interacting protein may be expressed naturally or may be recombinantly expressed; the candidate agent may be added exogenously, or be expressed naturally or recombinantly. In order to establish whether the interaction is with one or both WW domains, a competitive binding assay could be utilized, as described in Inglis et al., 2004.

In another embodiment, agents that modulate the interaction between an FBP21 polypeptide and another agent, for example but without limitation a protein, may be identified in a cell-based assay by contacting cells expressing an FBP21 polypeptide in the presence of a known interacting agent and a candidate modulating agent and selecting the candidate agent which modulates the interaction. Alternatively, agents that modulate an interaction between an FBP21 polypeptide and another agent, for example but without limitation a protein, may be identified in a cell-free assay system by contacting the polypeptide with an agent known to interact with the polypeptide in the presence of a candidate agent. A modulating agent can act as an antibody, a cofactor, an inhibitor, an activator or have an antagonistic or agonistic effect on the interaction between an FBP21 polypeptide and a known agent. As stated above the ability of the known agent to interact with an FBP21 polypeptide can be determined by methods known in the art. These assays, whether cell-based or cell-free, can be used to screen a plurality (e.g. a library) of candidate agents.

In another embodiment, a cell-based assay system is used to identify agents capable of modulating (i.e. stimulating or inhibiting) the activity of an FBP21 polypeptide. Accordingly, the activity of an FBP21 polypeptide is measured in a population of cells that naturally or recombinantly express an FBP21 polypeptide, in the presence of a candidate agent. Preferably, the activity of an FBP21 polypeptide is compared to a reference range or control. In a preferred embodiment, the activity of an FBP21 polypeptide is measured in a first and second population of cells that naturally or recombinantly express an FBP21 polypeptide, in the presence of agent or absence of a candidate agent (e.g. in the presence of a control agent) and the activity of the FBP21 polypeptide is compared. The candidate agent can then be identified as a modulator of the activity of an FBP21 polypeptide based on this comparison. Alternatively, the activity of an FBP21 polypeptide can be measured in a cell-free assay system where the FBP21 polypeptide is either natural or recombinant. Preferably, the activity of an FBP21 polypeptide is compared to a reference range or control. In a preferred embodiment, the activity of an FBP21 polypeptide is measured in a first and second sample in the presence or absence of a candidate agent and the activity of the FBP21 polypeptide is compared. The candidate agent can then be identified as a modulator of the activity of an FBP21 polypeptide based on this comparison.

The activity of an FBP21 polypeptide can be assessed by detecting its effect on a downstream effectors, for example but without limitation, detecting the differential splicing of target proteins, detecting the level or activity of a second messenger (e.g. cAMP, intracellular Ca²⁺, diacylglycerol, IP3, etc.), or detecting a cellular response, for example, proliferation, differentiation or transformation where appropriate as known by those skilled in the art. The candidate agent can then be identified as a modulator of the activity of an FBP21 polypeptide by comparing the effects of the candidate agent to the control agent. Suitable control agents include PBS or normal saline.

In one embodiment, agents that modulate the expression of an FBP21 polypeptide (i.e. up-regulate or down-regulate) are identified in a cell-based assay system. Accordingly, a population of cells expressing an FBP21 polypeptide or nucleic acid are contacted with a candidate agent and the ability of the candidate agent to alter expression of the FBP21 polypeptide or nucleic acid is determined by comparison to a reference range or control. In another embodiment, a first and second population of cells expressing an FBP21 polypeptide are contacted with a candidate agent or a control agent and the ability of the candidate agent to alter the expression of the FBP21 polypeptide or nucleic acid is determined by comparing the difference in the level of expression of the FBP21 polypeptide or nucleic acid between the first and second populations of cells. In a further embodiment, the expression of the FBP21 polypeptide or nucleic acid in the first population may be further compared to a reference range or control. If desired, this assay may be used to screen a plurality (e.g. a library) of candidate agents. The cell, for example, can be of prokaryotic origin (e.g. E. coli) or eukaryotic origin (e.g. yeast or mammalian). Further, the cells can express an FBP21 polypeptide or nucleic acid endogenously or be genetically engineered to express an FBP21 polypeptide or nucleic acid. The ability of the candidate agents to alter the expression of an FBP21 polypeptide or nucleic acid can be determined by methods known to those of skill in the art, for example and without limitation, by flow cytometry, radiolabelling, a scintillation assay, immunoprecipitation, Western blot analysis or Northern blot analysis.

In another embodiment, agents that modulate the expression of an FBP21 polypeptide or nucleic acid may be identified in an animal model. Examples of suitable animals include, but are not limited to, mice, rats, rabbits, monkeys, guinea pigs, dogs and cats. Preferably, the animal used represents a model of a disease associated with angiogenesis. Accordingly, a first and second group of mammals are administered with a candidate agent or a control agent and the ability of the candidate agent to modulate the expression of the FBP21 polypeptide or nucleic acid is determined by comparing the difference in the level of expression between the first and second group of mammals. Where desired, the expression levels of the FBP21 polypeptides or nucleic acid in the first and second groups of mammals can be compared to the level of a FBP21 polypeptide or nucleic acid in a control group of mammals. The candidate agent or a control agent can be administered by means known in the art (e.g. orally, rectally or parenterally such as intraperitoneally or intravenously). Changes in the expression of a polypeptide or nucleic acid can be assessed by the methods outlined above. In a particular embodiment, a therapeutically effective agent can be identified by monitoring an amelioration or improvement in disease symptoms, to delay onset or slow progression of the disease, for example but without limitation, a reduction in tumour size. Techniques known to physicians familiar with diseases associated with angiogenesis can be used to determine whether a candidate agent has altered one or more symptoms associated with the disease.

One skilled in the art will also appreciate that an FBP21 polypeptide may also be used in a method for the structure-based design of an agent, in particular a small molecule which acts to modulate (e.g. stimulate or inhibit) the activity of said polypeptide, said method comprising:
1) determining the three-dimensional structure of said polypeptide;
2) deducing the three-dimensional structure within the polypeptide of the likely reactive or binding site(s) of the agent;
3) synthesising candidate agents that are predicted to react or bind to the deduced reactive or binding site; and
4) testing whether the candidate agent is able to modulate the activity of said polypeptide.
It will be appreciated that the method described above is likely to be an iterative process.

As discussed herein, agents which interact with an FBP21 polypeptide find use in the treatment and/or prophylaxis of diseases associated with angiogenesis.

Once an agent which is able to interact with an FBP21 polypeptide or nucleic acid or which is capable of modulating the interaction, expression or activity of an FBP21 polypeptide or nucleic acid has been identified, it may further be examined for its anti-angiogenic activity using any one or more of the assays known to a person of skill in the art. For example, but without limitation, said assays may include: in vitro assays and in vivo assays such as those described in Farinelle et al., 1998 and Auerbach et al., 2003.

Further or specific aspects of the invention include:
A screen wherein whether the determination of whether or not the candidate agent interacts with the FPB21 polypeptide or FBP21 polypeptide fragment is made by reference to a control agent known to interact with the FPB21 polypeptide or FBP21 polypeptide fragment;
A screen wherein the FBP21 polypeptide or FBP21 polypeptide fragment or the candidate agent are labelled so as to enable detection of an interaction between the polypeptide or polypeptide fragment and the candidate agent;
A screen wherein the FBP21 polypeptide or FBP21 polypeptide fragment and the candidate agent are contacted in a cell free system;
A screen wherein the FBP21 polypeptide or FBP21 polypeptide fragment is immobilised on a solid support;
A screen wherein the determination of interaction of the candidate agent to the FBP21 polypeptide or FBP21 polypeptide fragment is made in a competition assay employing a reference agent known to bind to the FBP21 polypeptide or FBP21 polypeptide fragment;
A method of screening for anti-angiogenic agents which comprises identifying agents which modulate the activity or expression of FBP21 said method comprising the following steps: (i) contacting FBP21 expressing cells with a candidate agent; and (ii) determining whether or not the candidate agent modulates the activity or expression of FBP21 by monitoring a modulation in a detectable downstream response;
A method of screening for anti-angiogenic agents which comprises identifying agents which modulate the activity or expression of FBP21 said method comprising the following steps: (i) administering to an experimental animal a candidate agent; and (ii) determining whether or not the candidate agent modulates the activity or expression of FBP21 by monitoring a modulation in a detectable downstream response in the experimental animal;
A method of screening for anti-angiogenic agents which comprises identifying agents which modulate the activity or expression of FBP21 said method comprising the following steps: (i) administering to experimental animals respectively a candidate agent and a reference agent; and (ii) determining whether or not the candidate agent modulates the level of expression of FBP21 by monitoring the level of expression in the animals administered with the candidate agent by reference to the level of expression in the animals administered with the reference agent; and
A method of treating diseases associated with angiogenesis by administering to a patient in need thereof an effective amount of an anti-antiogenic agent identified by means of a method of screening according to earlier mentioned aspects of the invention.

This invention further provides for the use of agents identified using the methods of the present invention that interact with, or modulate the expression or activity of an FBP21 polypeptide or nucleic acid for the treatments or prophylaxis of diseases associated with angiogenesis. Hereinafter, these agents that are suitable for use in treatment are referred to as “active agents”.

The term ‘treatment’ includes either therapeutic or prophylactic therapy. When a reference is made herein to a method of treating or preventing a disease or condition using a particular active agent or combination of agents, it is to be understood that such a reference is intended to include the use of that active agent or combination of agents in the preparation of a medicament for the treatment or prevention of the disease or condition.

Accordingly, the present invention provides a method for the prophylaxis and/or treatment of a disease associated with angiogenesis, which comprises administering to said subject a therapeutically effective amount of at least one active agent of the invention.

In a further embodiment the present invention provides a method of treatment of diseases associated with angiogenesis, said method comprising administering to a patient in need thereof a therapeutically effective amount of an active agent.

In one embodiment the active agent is not borrelidin or an analogue thereof.

“Patient” embraces human and other animal (especially mammalian) subjects, preferably human subjects. Accordingly the methods and uses of the active agents of the invention are of use in human and veterinary medicine, preferably human medicine.

The aforementioned active agents or a formulation thereof may be administered by any conventional method for example but without limitation they may be administered parenterally (including intravenous administration), orally, topically (including buccal, sublingual or transdermal), via a medical device (e.g. a stent), by inhalation, or via injection (subcutaneous or intramuscular). The treatment may consist of a single dose or a plurality of doses over a period of time.

Whilst it is possible for active agents to be administered alone, it is preferable to present it as a pharmaceutical formulation, together with one or more acceptable carriers. Thus there is provided a pharmaceutical composition comprising an active agent together with one or more pharmaceutically acceptable diluents or carriers. The diluents(s) or carrier(s) must be “acceptable” in the sense of being compatible with the active agent and not deleterious to the recipients thereof. Examples of suitable carriers are described in more detail below.

The active agents may be administered alone or in combination with other therapeutic agents. Co-administration of two (or more) agents may allow for significantly lower doses of each to be used, thereby reducing the side effects seen. There is also provided a pharmaceutical composition comprising a compound of the invention and a further therapeutic agent together with one or more pharmaceutically acceptable diluents or carriers.

In a further aspect, the present invention provides for the use of an active agent in combination therapy with a second agent for the treatment a disease associated with angiogenesis.

In one embodiment, an active agent is co-administered with another therapeutic agent for the treatment of cancer or B-cell malignancies preferred additional agents include, but are not limited to, methotrexate, leukovorin, adriamycin, prenisone, bleomycin, cyclophosphamide, 5-fluorouracil, paclitaxel, docetaxel, vincristine, vinblastine, vinorelbine, doxorubicin, tamoxifen, toremifene, megestrol acetate, anastrozole, goserelin, anti-HER2 monoclonal antibody (e.g. Herceptin™), capecitabine, raloxifene hydrochloride, EGFR inhibitors (e.g. Iressa®, Tarceva™, Erbitux™), VEGF inhibitors (e.g. Avastin™), proteasome inhibitors (e.g. Velcade™) or Glivec®. Additionally, an active agent may be administered in combination with other therapies including, but not limited to, radiotherapy or surgery.

The formulations may conveniently be presented in unit dosage form and may be prepared by any of the methods well known in the art of pharmacy. Such methods include the step of bringing into association the active agent with the carrier which constitutes one or more accessory ingredients. In general the formulations are prepared by uniformly and intimately bringing into association the active agent with liquid carriers or finely divided solid carriers or both, and then, if necessary, shaping the product.

The active agents will normally be administered orally or by any parenteral route, in the form of a pharmaceutical formulation comprising the active agent, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

For example, the active agents can be administered orally, buccally or sublingually in the form of tablets, capsules, ovules, elixirs, solutions or suspensions, which may contain flavouring or colouring agents, for immediate-, delayed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the active agents may be combined with various sweetening or flavouring agents, colouring matter or dyes, with emulsifying and/or suspending agents and with diluents such as water, ethanol, propylene glycol and glycerin, and combinations thereof.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations in accordance with the present invention suitable for oral administration may be presented as discrete units such as capsules, cachets or tablets, each containing a predetermined amount of the active agent; as a powder or granules; as a solution or a suspension in an aqueous liquid or a non-aqueous liquid; or as an oil-in-water liquid emulsion or a water-in-oil liquid emulsion. The active agent may also be presented as a bolus, electuary or paste.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active agent in a suitable liquid carrier.

It should be understood that in addition to the ingredients particularly mentioned above the formulations of this invention may include other components conventional in the art having regard to the type of formulation in question, for example those suitable for oral administration may include flavouring agents.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base.

Alternatively, the active agent may be formulated in a cream with an oil-in-water cream base or a water-in-oil base.

For parenteral administration, fluid unit dosage forms are prepared utilizing the active agent and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as local anaesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The active agents may also be administered using medical devices known in the art. For example, in one embodiment, a pharmaceutical composition of the invention can be administered with a needleless hypodermic injection device, such as the devices disclosed in U.S. Pat. No. 5,399,163; U.S. Pat. No. 5,383,851; U.S. Pat. No. 5,312,335; U.S. Pat. No. 5,064,413; U.S. Pat. No. 4,941,880; U.S. Pat. No. 4,790,824; or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The dosage to be administered of an active agent will vary according to the particular compound, the disease involved, the subject, and the nature and severity of the disease and the physical condition of the subject, and the selected route of administration. The appropriate dosage can be readily determined by a person skilled in the art.

The compositions may contain from 0.1% by weight, preferably from 5-60%, more preferably from 10-30% by weight, of a compound of invention, depending on the method of administration.

It will be recognized by one of skill in the art that the optimal quantity and spacing of individual dosages of an active agent will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

### BRIEF DESCRIPTION OF THE FIGURES

FIG. 1A: Structure of borrelidin (1) and pre-borrelidin (2)

FIG. 1B: Structure of biotinylated borrelidin (3), biotinylated pre-borrelidin (4) and two borrelidin analogues (5) and (6)

FIG. 2A Nucleic acid sequence of FBP21 (SEQ ID NO: 1)

FIG. 2B: Amino acid sequence of FBP21 (SEQ ID NO: 2). Sequence corresponding to the two WW domains is shown in bold.

FIG. 3: Alignment of cDNA phage insert and FBP21. Phage backbone sequence shown in bold. Flanking EcoRI/HindIII sites underlined.

FIG. 4: Alignment of predicted displayed protein and FBP21. WW domains highlighted in bold.

FIG. 5: A gel image showing the PCR products generated using the eluted phage as template.

FIG. 6: (A) ELISA for VEGFₓₓₓ showing significant downregulation of VEGFₓₓₓ by compound 6, compound 5 and borrelidin.

(B) ELISA for VEGF_{xxxb} showing significant upregulation of VEGF_{xxxb} by compound 6, compound 5 and borrelidin.

FIG. 7: Comparison of the effect of compound 5 on the VEGF levels by ELISA. RPE cells were treated with increasing concentrations of compound 5 for 24 hours and the VEGFxxxb and total VEGF concentrations measured by ELISA. **p<0.05, Dunnett's, **p<0.01, 95% confidence interval, alpha=0.05, N=3.

FIG. 8: (A) Western Blot analysis of the cell lysate for expression of VEGF165b. Primary antibody: 56/1 mouse anti-h antibody (200 μg/ml, 1:250 dilution) and secondary antibody: Goat anti-mouse antibody (1:7000 dilution).

(B) Western Blot analysis of the cell lysate for expression of β-Actin. Primary antibody: goat anti-human β-actin IgG (0.25 μg/ml). Secondary antibody: rabbit anti-goat IgG (1:10000 dilution).

FIG. 9: Plasmid restriction maps for pOriGene-FBP21 and pWV327 showing position of main genetic elements

FIG. 10: Comparison of the effect of compound 5 on RPE and HEK cells transfected with either pOriGene-FBP21 (control) or pWV327 (FBP21)

FIG. 11: Images of mouse retinas stained with Isolectin, showing levels of angiogenesis when treated with either vehicle, 0.5 uM or 5 uM or compound 5.

FIG. 12: Comparison of percentage neovascularization in contralateral (untreated), vehicle treated and 0.5 or 5 μM compound 5 treated retinas. Filled boxes show average neovascurization, filled circles show neovascularization in individual animals.

FIG. 13: (A) Average percentage neovascularization for each of the treatment groups in either the contralateral or untreated retinas.

(B) Percentage neovascularization relative to control in all treatments groups.

### EXAMPLES

### General Methods

### Phage Display and Biopanning

Streptavidin wells were incubated with 1 μM biotinylated probe at 4° C. overnight to prepare the wells for biopanning. After incubation, unbound biotinylated probe was removed from the well by washing with phosphate buffered saline (PBS). Wells were then blocked with blocking solution (3% skimmed milk in PBS) for at least one hour at room temperature. Blocked wells were then briefly washed with PBS and were ready for biopanning use.

Novagen T7 phage libraries were amplified using standard techniques (see Novagen T7Select™ manual provided with Novagen phage library) and stored either in 0.5 M NaCl at 4° C. (for short term storage) or in 8% glycerol at −80° C. (for long term storage).

Amplified phage libraries were tittered using standard techniques and were found to contain an average of 5×10¹⁰ plaque forming units per mL (pfu/mL). In the first round of biopanning, 0.15 mL of amplified phage library was added to an empty streptavidin coated well and incubated at room temperature for at least 1 hour. This step was included to deselect any phage displaying streptavidin binding proteins. After incubation, the phage were removed from the well and mixed with 50 μL 4× blocking solution (12% skimmed milk in PBS) and incubated at room temperature for at least one hour.

0.125 mL of blocked phage library was added to each pre-prepared biotinylated well and incubated at room temperature for at least one hour. The supplied library contains an estimated 1.7×10⁷ distinct primary recombinant phage clones so >200 copies of each clone were added to the well.

After incubation, unbound phage were removed from the well with wash solution. The wash solution was varied depending on the round of biopanning. For first round of biopanning PBS1 was used. For subsequent rounds the stringency of the wash was increased by using sequentially PBS2, PBS3 and PBS4 (see Table I).

During the first biopanning round, ten washes were performed with PBS1 followed by five washes with PBS to remove all traces of Tween20 and NaCl from the well. Similarly for subsequent rounds ten washes were performed with the appropriate wash solution followed by 5 washes with PBS.

TABLE I

| | | |
|---|---|---|
| Round 1 | PBS1 | 150 mM NaCl, 0.1% Tween20 in PBS |
| Round 2 | PBS2 | 170 mM NaCl, 0.3% Tween20 in PBS |
| Round 3 | PBS3 | 200 mM NaCl, 0.5% Tween20 in PBS |
| Round 4 | PBS4 | 250 mM NaCl, 0.7% Tween20 in PBS |

Two different elution techniques were employed to remove bound phage from the wells.
i) SDS elution: 0.1 mL SDS elution solution (1% SDS in PBS) was added to the well and incubated at room temperature for at least one hour.
ii) E. coli elution (McKenzie, 2004): 0.1 mL E. coli elution solution (the host E. coli strain (BLT5403) grown to OD=0.4) was added to the well and incubated at room temperature for at least one hour.

The eluent was removed from the well and pipetted into 50 mL E. coli BLT5403 in 2xTY OD=0.4 in a 250 mL conical flask. The flask was incubated at 37° C. with shaking at 250 rpm for approximately four hours or until complete lysis had occurred. The lysate was transferred to a 50 mL falcon tube, NaCl was added to a final concentration of 0.5 M, and the lysate was centrifuged at 4000 rpm for 10 minutes. The supernatant was transferred to a new 50 mL tube and was stored at 4° C. or used in a subsequent round of biopanning.

### Production of Borrelidin and Pre-Borrelidin

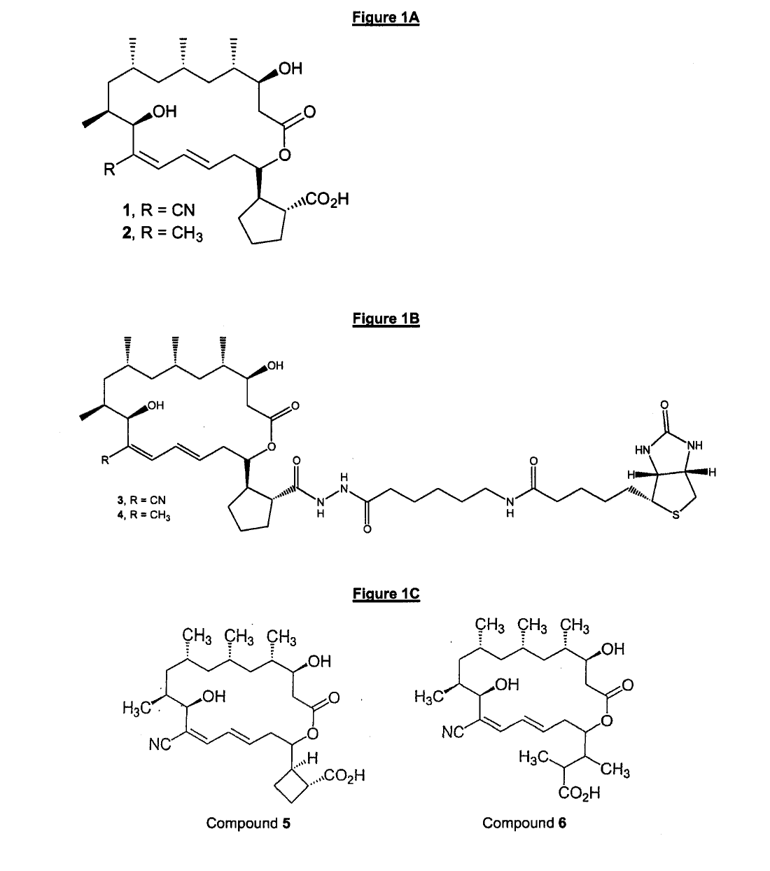

Borrelidin for use in the methods described below may be produced by any methods known to a person of skill in the art. In particular, it may be obtained by fermentation from a suitable producer strain, e.g. Streptomyces rochei ATCC23956, Streptomyces parvulus Tü113 or Streptomyces parvulus Tü4055, using processes well known to a person of skill in the art. A suitable process is described in JP 9-227,549. Analogues of borrelidin may be produced according to the methods described in WO 2004/058976 or in JP 9-227,549, specifically the production of pre-borrelidin was performed as described in Olano et al., 2004b, and the production of compound 5, 17-des-(cyclopentane-2′-carboxylic acid)-17-(cyclobutane-2′-carboxylic acid) borrelidin (described as compound 18, in example 33 in WO 2004/058976, and as compound 4 in Moss et al., 2006) and compound 6, (generated by feeding 2,3-dimethyl succinic acid in a similar manner to example 33 in WO 2004/058976, also described as compound 5 in Moss et al., 2006) are detailed in WO 2004/058976 and Moss et al., 2006, and the structures shown in FIG. 1C.

Alternatively, borrelidin may be produced via total synthesis as described Hanessian et al., 2003; Duffey et al., 2003; Nagamitsu et al., 2004; or Vong et al., 2004.

### Example 1

### Synthesis of Biotinylated Borrelidin and Pre-Borrelidin

### 1.1: Synthesis of Biotinylated Borrelidin (C6 Spacer Linked)

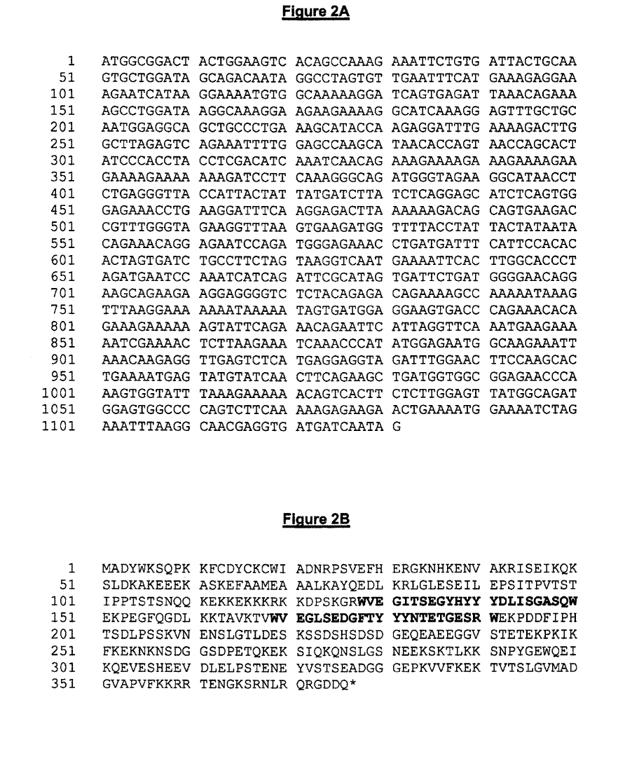

A sample of borrelidin (10 mg, 0.02 mmol) was dissolved into tetrahydrofuran (THF) (2 mL) and cooled to −15° C. in an ice/salt bath. Isobutyl chloroformate (0.3 mL, 2.4 mmol) and triethylamine (0.32 mL, 2.3 mmol) were dissolved in THF (10 mL) and an aliquot of this mixture (0.1 mL) was added to the borrelidin solution. After stirring for 30 min the solution was filtered to remove triethylamine hydrochloride. To the resulting solution was added a mixture of biotinamidohexanoic acid hydrazide (Sigma, 36 mg) dissolved in THF (2 mL) and water (2 mL). After 2 hours stirring at room temperature the mixture was diluted with water (to 25 mL total volume) and passed through a C18 Bond Elute cartridge (5 g). This was washed with water (20 mL) and then eluted with methanol (20 mL). Analysis by LCMS showed that ˜95% of the borrelidin had been converted to the biotinylated form (3). The methanol solution was evaporated to dryness to yield a clear oil (16.5 mg). LCMS: m/z=841.5 [M-H]⁻, see FIG. 2.

### 1.2: Synthesis of Biotinylated Pre-Borrelidin (C6 Spacer Linked)

A sample of pre-borrelidin (10 mg, 0.02 mmol) was reacted in a manner identical to that described in Example 1.1 above. After 2 hours stirring at room temperature the mixture was diluted with water (to 25 ml total volume) and passed through a C18 Bond Elute cartridge (5 g). This was washed with water (20 mL) and then eluted with methanol (20 mL). Analysis by LCMS showed that ˜95% of the pre-borrelidin had been converted to the biotinylated form (4). The methanol solution was evaporated to dryness to yield a clear oil (6 mg). LCMS: m/z=830.5 [M-H], see FIG. 2⁻.

### Example 2

### Biological Activity of Borrelidin and Pre-Borrelidin

Angiogenesis assays were provided by the NCI, details of the assay protocols are given below. In each assays TNP-470 and paclitaxel (Taxol®) were used as reference compounds.

### Growth Inhibition Assay

HUVEC (1.5×10³) were plated in a 96-well plate in 100 μL of EBM-2 (Clonetic #CC3162). After 24 h (day 0), the test compound (100 μL) was added to each well at 2 times the desired concentration (5-7 concentration levels) in EBM-2 medium. On day 0, one plate was stained with 0.5% crystal violet in 20% methanol for 10 minutes, rinsed with water, and air-dried. The remaining plates were incubated for 72 h at 37° C. After 72 h, plates were stained with 0.5% crystal violet in 20% methanol, rinsed with water and air-dried. The stain was eluted with 1:1 solution of ethanol:0.1 M sodium citrate (including day 0 plate), and absorbance was measured at 540 nm with an ELISA reader (Dynatech Laboratories). Day 0 absorbance was subtracted from the 72 h plates and data was plotted as percentage of control proliferation (vehicle treated cells). The IC₅₀ (drug concentration causing 50% inhibition) was calculated from the plotted data.

### Cord Formation Assay:

Matrigel (60 μL of 10 mg/mL; Collaborative Lab #35423) was placed in each well of an ice-cold 96-well plate. The plate was allowed to sit at room temperature for 15 minutes then it was incubated at 37° C. for 30 minutes to permit the matrigel to polymerize. In the mean time, HUVEC were prepared in EGM-2 (Clonetic #CC3162) at a concentration of 2×10⁵ cells/mL. The test compound was prepared at 2 times the desired concentration (5 concentration levels) in the same medium. The cells (500 μL) and the drug at 2 times concentration (500 μL) were mixed and 200 μL of this suspension was placed in duplicate on the polymerized matrigel. After 24 h incubation, triplicate pictures were taken for each concentration using a Bioquant Image Analysis system. Drug effect (IC₅₀) is assessed compared to untreated controls by measuring the length of cords formed and number of junctions.

### Cell Migration Assay

Migration was assessed using the 48-well Boyden chamber and 8 μm pore size collagen-coated (10 μg/mL rat tail collagen; Collaborative Laboratories) polycarbonate filters (Osmonics, Inc.). The bottom chamber wells received 27-29 μL of DMEM medium alone (baseline) or medium containing chemo-attractant (bFGF, VEGF or Swiss 3T3 cell conditioned medium). The top chambers received 45 μL of HUVEC cell suspension (1×10⁶ cells/mL) prepared in DMEM+1% BSA with or without test compound. After 5 h incubation at 37° C., the membrane was rinsed in PBS, fixed and stained in Diff-Quick solutions. The filter was placed on a glass slide with the migrated cells facing down and cells on top were removed using a Kimwipe. The testing was performed in 4-6 replicates and five fields were counted from each well. Negative, unstimulated, control values were subtracted from stimulated control and drug treated values and data was plotted as mean migrated cell±S.D. The IC₅₀ was calculated from the plotted data.

TABLE II
results of antiangiogenic assays.

| | Assay, IC₅₀ (nM) | | |
|---|---|---|---|
| Compound | Growth Inhibition | Cord Formation* | Chemotaxis |
| Borrelidin | 3.25 | 46.5 | 63 |
| Pre-borrelidin | 2380 | >30,000 | 461 |
| TNP470 | 3.16 ± 0.82 | 1000 | 500 |
| Taxol | 1.65 ± 0.12 | 50 | 100 |

### Example 3

### 3.1 Biopanning Rounds Using Borrelidin as Bait

Four rounds of biopanning were performed using the method described above using the Novagen T7Select Human Colon Tumour Library. In each round, the SDS elution technique was used. The amplified lysate from round four was used as the input phage for two fifth round biopanning experiments that were performed in parallel, one using SDS elution and the other using E. coli elution.

Lysates from the fifth round were serially diluted, mixed with 0.25 mL of E. coli culture (BLT5403 grown overnight at 37° C.), mixed with 3 mL melted top agarose (pre-equilibrated to 45° C.) and poured onto a pre-warmed plate of 2TY agar. Plates were incubated at 37° C. for 2-4 hours until a bacterial lawn containing phage plaques was seen. 12 individual phage plaques from each round 5 experiment were cut from the plates and placed into tubes containing 0.2 mL PBS. Tubes were vortexed briefly to liberate the phage from the agar plug. 1 μL of each phage plaque suspension was used as the template in a PCR reaction using T7SelectUP GGAGCTGTCGTATTCCAGTC (SEQ ID NO: 3) and T7SelectDOWN AACCCCTCAAGACCCGTTTA (SEQ ID NO: 4) primers, Taq DNA polymerase and the following PCR program:

1 Cycle:

80° C. for 10 minutes

30-35 cycles:

94° C. for 1 minute

55° C. for 1 minute

72° C. for 2 minutes

PCR products were cleaned and sequenced with T7SelectUP and T7SelectDOWN and the resultant data were submitted for nucleotide BLASTN searches on the NCBI website. Predicted displayed amino acid sequences were submitted to BLASTP.

### 3.2. Results

The sequences of all twelve single plaques were analysed from each elution method.

### 3.2.1 SDS Elution Method

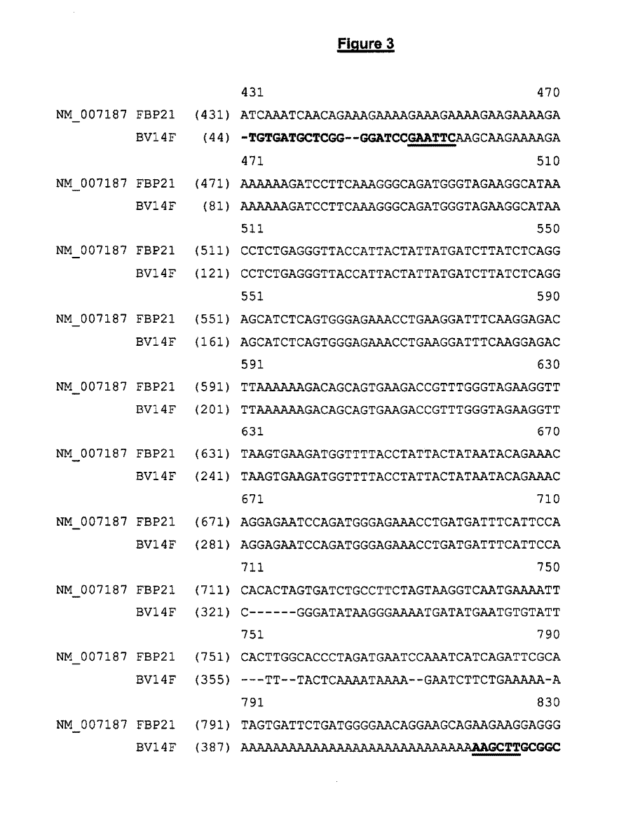

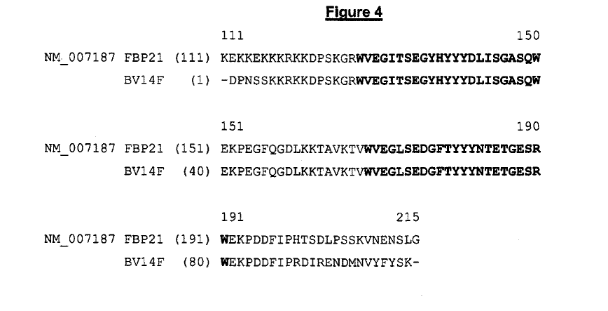

Sequence analysis showed that two out of twelve of the SDS eluted phage contained a cDNA insert which shared 100% homology over a 250 by region with Human Formin Binding Protein 21 (FBP21, the nucleic acid sequence of which is shown as FIG. 2A (SEQ ID NO: 1) and the amino acid sequence of which is shown as FIG. 2B (SEQ ID NO: 2)) (FIG. 3). The predicted displayed amino acid sequences of the clones were analysed and the two clones encode 99 amino acids in frame with the phage coat protein of which a segment of 83 residues share 100% homology with FBP21 (FIG. 4). This 83 amino acid protein fragment contains two WW domains and is therefore likely to encode the active site(s) of the enzyme.

Sequence data from the remaining 10 out of 12 phage did not produce any significant hits. The construction of the original library is carried out such that some DNA sequences are in-frame and some are not. Only in-frame sequences will result in a protein and only sequences that could result in a protein were considered. DNA sequences that could not result in a protein (e.g. they are reversed with respect to the direction of expression, or they result in a protein that is not in-frame with the upstream protein) were not considered. The majority of clones displayed less than 20 amino acids in frame and were disregarded as background binders (Jin et al., 2002 February; 9(2):157-62)

3.2.2 E. coli Elution Method

Sequence analysis showed that eleven out of twelve of the SDS eluted phage contained a cDNA insert which shared 100% homology over a 250 by region with Human Formin Binding Protein 21 (FBP21) and was identical to the cDNA inserts from the two SDS eluted samples described above (FIG. 3).

The remaining clone contained a cDNA insert that was under 150 by in length and the sequence BLAST generated no hits.

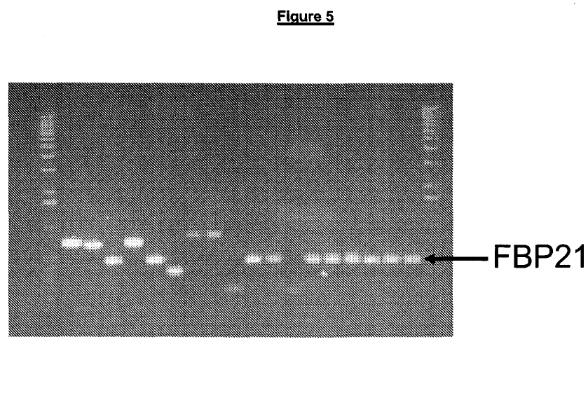

FIG. 5 shows a gel image containing the PCR products using the eluted phage as the template. In the image lanes 1-9 contain SDS eluted phage and lanes 10-18 contain E. coli eluted phage

### 3.3—Biopanning Repeats

The experiment described above was repeated using the same input library and same methodology as described above. Five rounds of SDS elution were performed, followed by two subsequent rounds using E. coli elution. Results were as follows

### 3.3.1 SDS Elution Method

Sequence analysis showed that 1 out of 15 inserts encoded FBP21, an identical fragment to the one pulled out in the first experiment. 1 out of 15 inserts encoded Serine/Arginine splicing factor SFRS11, 2 out of 15 inserts encoded SMG-1 PI-3-kinase related kinase and the remaining clones did not match anything significant in BLAST search.

3.3.2 E. coli Elution Method

Sequence analysis showed that 12 out of 14 inserts encoded FBP21, an identical fragment to the one pulled out in the example 1.2 and from SDS elution in the example 1.3.1. The remaining clones did not match anything significant in BLAST search.

### Example 4

### Biopanning Using Biotinylated Pre-Borrelidin as Bait

In parallel with the biopanning described in Example 2, identical experiments were performed using biotinylated pre-borrelidin as biopanning bait. Experiment was repeated using the same input library and same methodology as described above.

### 4.1 SDS Elution Method

Sequence analysis showed that none of the 13 inserts encoded FBP21. 2 out of 13 inserts encoded SMG1 PI-3-kinase related kinase and the remaining clones did not match anything significant in BLAST search or encoded out of frame proteins.

4.2 E. coli Elution Method

Sequence analysis showed that none of the 13 inserts encoded FBP21, 6 out of 18 inserts encoded SMG1 PI-3-kinase related kinase and the remaining clones did not match anything significant in BLAST search or encoded out of frame proteins.

These results demonstrate that although the specificity varies depending on the elution method used (McKenzie et al., 2004), biopanning using borrelidin consistently identified an FBP21 fragment. In contrast, this fragment was never identified using the inactive pre-borrelidin as a negative control. These data indicate that FBP21 is a target of borrelidin.

### Example 5

### Treatment of RPE Cell Lines with Borrelidin Analogues Effects VEGFₓₓₓb Levels

### 5.1 Effect of Borrelidin Analogues on VEGF in Retinal Pigmented Epithelial (RPE) Cell Lines

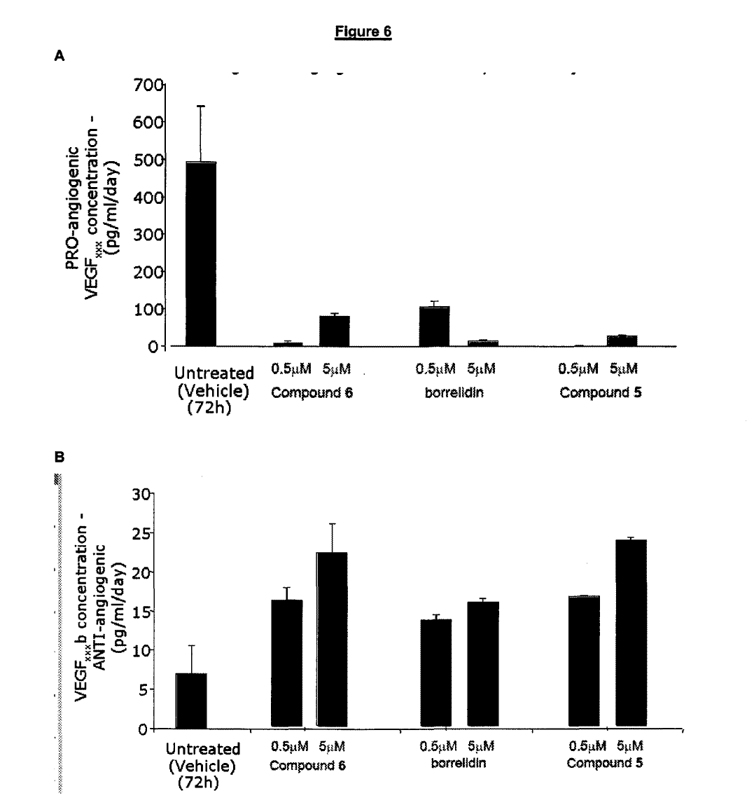

To determine whether borrelidin and analogues can alter VEGF splicing between the two families of isoforms, RPE cells, known to express both families of isoforms, were treated with 0.5 μM or 5 μM of three borrelidin compounds, compound 5, compound 6 and borrelidin. Media was sampled after 72 hours treatment, and assessed for total VEGF and VEGFₓₓₓb expression (see Bates et al., 2002, Woolard et al., 2004 and Varey et al., 2008 for methods). FIGS. 6A and B show that all three borrelidins downregulate angiogenic VEGFₓₓₓ expression, and upregulate anti-angiogenic VEGF_{xxxb} expression in RPE cells.

### 5.1 Dose Dependent Effect of Compound 5 on VEGF in RPE Cell Lines

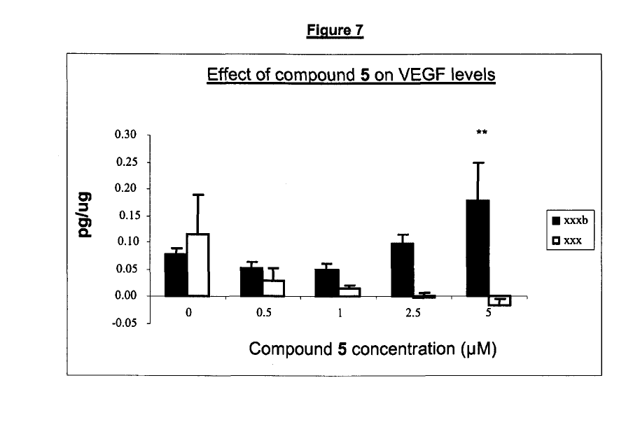

RPE cell lysate was extracted after 24 hours incubation with increasing concentrations of compound 5. VEGF_{xxxb} and VEGFₓₓₓ levels were then analysed by ELISA (see Bates et al., 2002, Woolard et al., 2004 and Varey et al., 2008 for methods). These levels were compared to total cell protein levels. The results (FIG. 7) showed a dose dependent decrease in total VEGF levels and a significant effect on VEGF_{xxxb} levels. At 5 μM the increase in VEGF_{xxxb} levels was seen to be statistically higher than those of the control (with no compound 5 treatment).

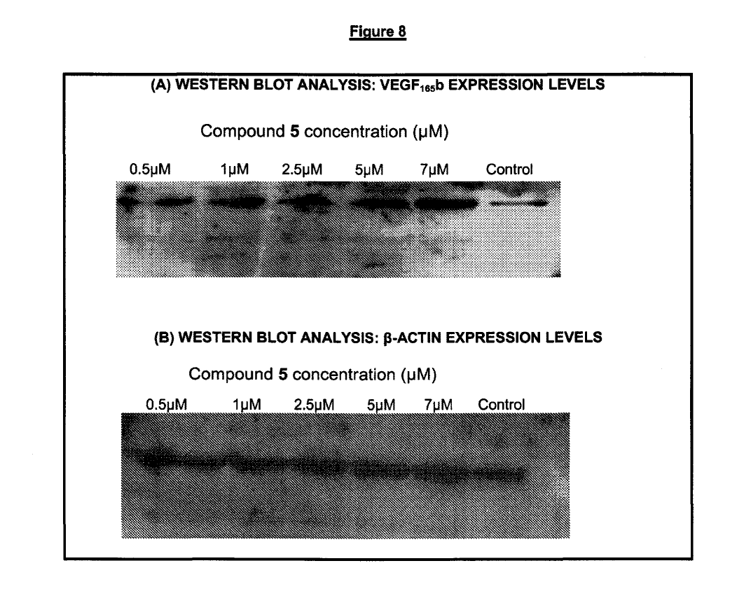

Western blot analysis also showed a similar effect on VEGF_{xxxb} levels (FIG. 8)

This data shows that compound 5 differentially affects the splice isoforms of VEGF.

### Example 6

### Transfection of RPE and HEK Cells with FBP21 and Effect on Compound 5 Treatment

Borrelidin derivatives act to increase distal splicing of VEGF through FBP21

### 6.1 Generation of a Contruct Expressing FBP21

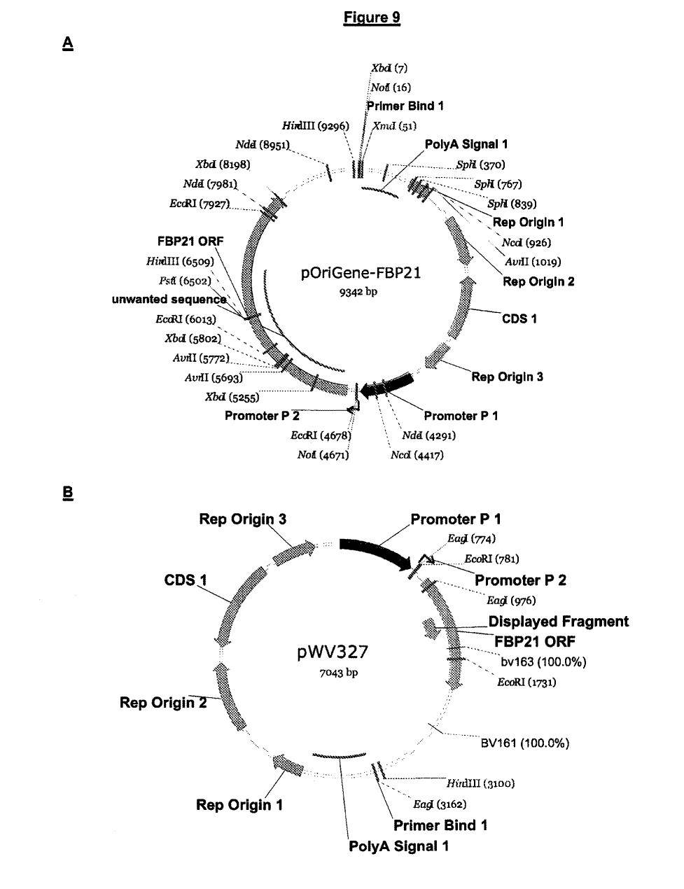

pOriGene-FBP21 (FIG. 9A, SEQ ID No. 5) was purchased from Origene. This construct contains the cDNA for the human FBP21 gene. Due to the presence in this contruct of non-essential DNA which would have disrupted the expression of FBP21, a three part ligation was carried out to remove this DNA. This generated pWV327 (FIG. 9B, SEQ ID No. 6).

6.2 Transfection of RPE and HEK Cells with a Contruct Expressing FBP21

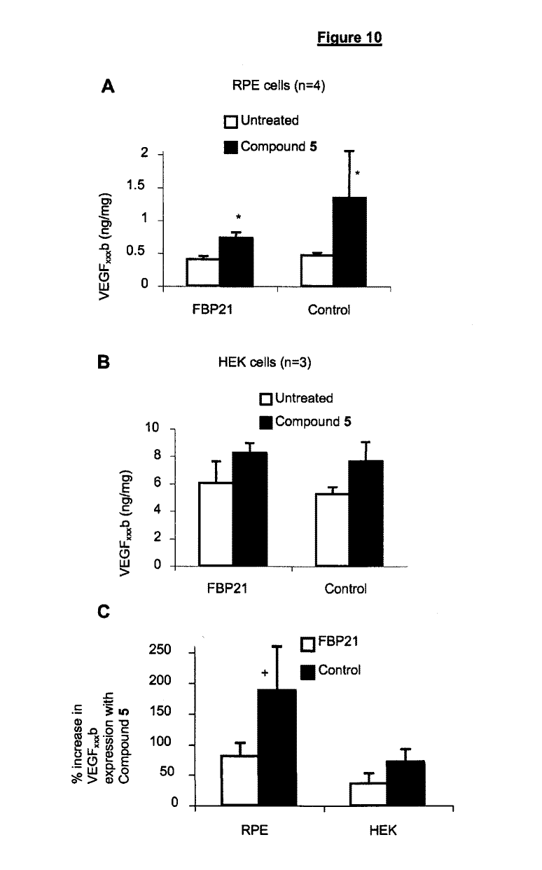

Both RPE and HEK cells were transfected with either pOriGene-FBP21 (as a control, due to the lack of FBP21 expression) or pWV327 (for overexpression of FBP21), using standard techniques. These cell lines were then treated with compound 5 (7 uM) or untreated, and VEGF_{xxxb} levels analysed after 24 hours. FIGS. 10A, B and C show the results of this analysis. In both cases, FBP21 transfection reduced the effect of compound 5, as anticipated for an compound which functions through an effect on FBP21.

### Example 7

### Evidence of In Vivo Effect on Angiogenesis

Evidence that compound 5, a borrelidin analogue which affects splicing of VEGF through FBP21, affects angiogenesis was gained from an in vivo mouse model, Retinopathy of Maturity (ROP), which mimics some of the processes in Age-related Macular Degeneration and other eye diseases where neovascularisation is associated with disease progression.

### 7.1 Retinopathy of Maturity In Vivo Angiogenesis Model

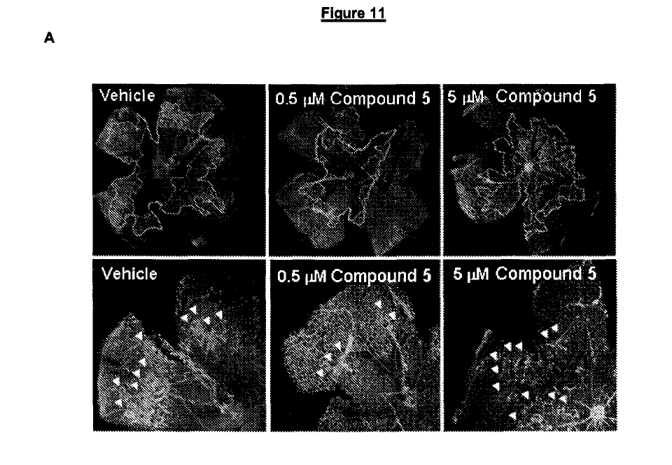

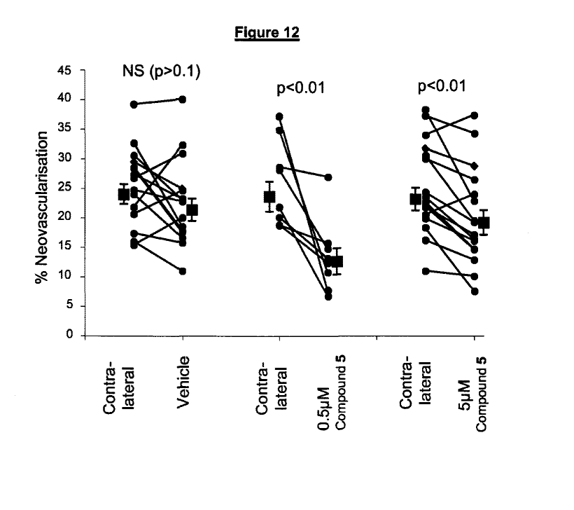

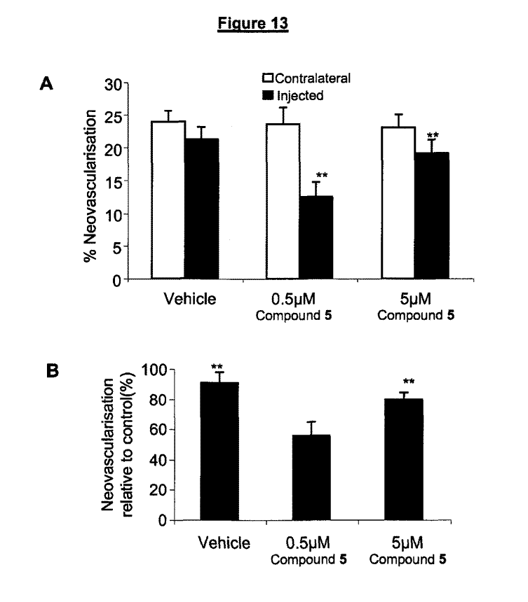

Compound 5 was dosed at two concentrations, 0.5 μM and 5 μM, in a mouse Retinopathy of Prematurity (ROP) model (see Konopatskaya et al., 2006 for methods). Oxygen induced retinopathy was induced by placing the mice at 75% oxygen from day 7 to day 12 post birth and then removing them to normal room air. Mice were injected with either vehicle (0.02% DMSO in HBSS) or 0.5 μM or 5 μM compound 5 into one eye, and the other left uninjected (contralateral). At day 17 mice were killed, retinas removed and stained for endothelial cells with isolectin (FIG. 11). Quantification revealed a significant reduction in neovascularixation by 0.5 μM and 5 μM compound 5 (p<0.01 by paired t-test) compared with contralateral (see FIGS. 12 and 13). Comparison of injection regimes showed that 0.5 μM significantly inhibited the percentage neovascularisation compared to both vehicle and the 5 μM dose.

### REFERENCES

Auerbach, R., R. Lewis; B Shinners, L. Kubai and N. Akhtar; (2003) Angiogenesis Assays: A critical Overview; Clinical Chemistry, 49 (1), 32-40.
Bartel, P., Chien, C.-T., Sternglanz, R. & Fields, S. Elimination of false positives that arise in using the two-hybrid system. BioTechniques 14, 920-924 (1993).
Bates, D. O., Cui, T-G., Doughty, J. M., Winkler, M., Sugiono, M., Shields, J. D., Peat, D., Gilliatt, D., and Harper, S. J. (2002). VEGF₁₆₅b, an inhibitory variant of vascular endothelial growth factor, is down-regulated in renal cell carcinoma. Cancer Res., 62, 4123-4131.

Bedford, M. T., Reed, R., and Leder, P. (1998) WW domain-mediated interactions reveal a spliceosome-associated protein that binds a third class of proline-rich motif: the proline glycine and methionine rich motif. Proc. Nat. Acad. Sci. USA, 95, 10602-10607.
Bedford M T, Chan D C, Leder P (1997) FBP WW domains and the Abl SH3 domain bind to a specific class of proline-rich ligands The EMBO Journal 16(9):2376-2383
Bedford M T, Reed R, Leder P (1998) WW domain-mediated interactions reveal a spliceosome-associated protein that binds a third class of proline-rich motif: The proline glycine and methionine-rich motif. Proc. Natl. Acad. Sci. USA 95:10602-10607
Berger, J., Jampolsky, L. M., and Goldberg, M. W. (1949) Borrelidin, a new antibiotic with anti-Borrelia activity and penicillin enhancement properties. Arc. Biochem. 22:476-478.
Blackstock, W. & Weir, M. 1999, Proteomics: quantitative and physical mapping of cellular proteins. Trends in Biotechnology, 17(3):121-7.
Caligiuri, M., Becker, F., Murthi, K., Kaplan, F., Dedier, S., Kaufman, C., Machl, A., Zybarth, G., Richard, J., Bockovich, N., Kluge, A., and Kley, N. (2005) A proteome-wide CDK/CRK-specific kinase inhibitor promotes tumour cell death in the absence of cel cycle progression. Chem. Biol. 12,1103-1115.
Carell, T., Wintner, E. A. and Rebek, J., Jr. (1994) A solution-phase screening procedure for the isolation of active compounds from a library of molecules. Angew. Chem. Int. Ed. Engl. 33: 2061-2064.
Carell, T., Wintner, E. A., Bashir-Hashemi, A. and Rebek, Jr. (1994) A novel procedure for the synthesis of libraries containing small organic molecules. Angew. Chem. Int. Ed. Engl. 33: 2059-2061.
Carmeliet, P. (2005) Angiogenesis in life, disease and medicine. Nature, 438, 932-936.
Chan D C, Bedford M T, Leder P (1996) Formin binding proteins bear WWP/WW domains that bind proline-rich peptides and functionally resemble SH3 domains The EMBO Journal 15(5):1045-1054
Chan D C and Leder P (1996) Genetic evidence that formins function within the nucleus. 271(38):23472-23477
Cho C Y, Moran E J, Cherry S R, Stephans J C, Fodor S P, Adams C L, Sundaram A, Jacobs J W, Schultz P G. (1993), Science 261(5126):1303-5;
Cull M G, Miller J F, and Schatz P J, 1992, Screening for Receptor Ligands Using Large Libraries of Peptides Linked to the C Terminus of the lac Repressor Proc. Natl. Acad. Sci; 89: 1865-1869.
Cwirla S E, Peters E A, Barrett R W, Dower W J. (1990) Peptides on phage: a vast library of peptides for identifying ligands. Proc Natl Acad Sci USA.;87(16):6378-6382
Devlin J J, Panganiban L C, Devlin P E (1990). Random peptide libraries: a source of specific protein binding molecules. Science.; 249(4967):404-406
DeWitt S H, Kiely J S, Stankovic C J, Schroeder M C, Cody D M, Pavia M R (1993) “Diversomers”: an approach to nonpeptide, nonoligomeric chemical diversity. Proc. Natl. Acad. Sci. USA 90: 6909 -13;
Dickinson, L., Griffiths, A. J., Mason, C. G., and Mills, R. F. (1965) Anti-viral activity of two antibiotics isolated from a species of Streptomyces. Nature 206:265-268.
Duffey, M. O., A. LeTiran, et al. (2003) Enantioselective total synthesis of borrelidin. J Am Chem Soc 125(6): 1458-1459.
Eastwood, E. L. and S. E. Schaus (2003) Borrelidin induces the transcription of amino acid biosynthetic enzymes via a GCN4-dependent pathway. Bioorg Med Chem Lett 13(13): 2235-7.

Erb E, Janda K D, and Brenner S (1994) Recursive deconvolution of combinatorial chemical libraries. Proc. Natl. Acad. Sci. USA 91: 11422
Farinelle, S., C. Dehauwer, F. Darro, C. Decaestecker, J. Fontaine, J-L Pasteels, P. Van Ham, G Atassi, and R. Kiss (1998), Setting up of an original computer-assisted methodology to characterize in vitro drug-induced anti-angiogenic effects. Int J Mol Med. 2(5):545-553
Felici F, Castagnoli L, Musacchio A, Jappelli R, and Cesareni G (1991) Selection of antibody ligands from a large library of oligopeptides expressed on a multivalent exposition vector. J Mol Biol. November 20; 222(2):301-310.
Ferrara, N., and Davis-Smyth, T. (1997). The biology of vascular endothelial growth factor. Endocr. Rev., 18, 4-25.
Ferrara, N., and Kerbel, R. S. (2005) Angiogenesis as a therapeutic target. Nature, 438, 967-974.
Fodor, 1993, Nature 364: 555-556
Folkman, J. (1986) How is blood vessel growth regulated in normal and neoplastic tissue? G.H.A. Cloves Memorial Lecture. Cancer Res. 51:467-473.
Funahashi, Y., Wakabayashi, T., Semba, T., Sonoda, J., Kitoh, K., and Yoshimatsu, K. (1999) Establishment of a quantitative mouse dorsal air sac model and its application to evaluate a new angiogenesis inhibitor. Oncol. Res. 11:319-329.
Gallop M A, Barrett R W, Dower W J, Fodor S P, Gordon E M. (1994) Applications of combinatorial technologies to drug discovery. 1. Background and peptide combinatorial libraries. J. Med. Chem. 37(9):1233-51.
Gavin A C, Bosche M, Krause R, Grandi P, Marzioch M, Bauer A, Schultz J, Rick J M, Michon A M, Cruciat C M, et al.: 2002 Functional organization of the yeast proteome by systematic analysis of protein complexes. Nature, 415:141-147
Rigaut G, Shevchenko A, Rutz B, Wilm M, Mann M & Séraphin B, (1999) A generic protein purification method for protein complex characterization and proteome exploration Nature Biotechnology 17, 1030-1032
Hanessian, S., Y. Yang, et al. (2003). Application of conformation design in acyclic stereoselection: total synthesis of borrelidin as the crystalline benzene solvate. J Am Chem Soc 125(45): 13784-92.
Ho Y, Gruhler A, Heilbut A, Bader GD, Moore L, Adams SL, Millar A, Taylor P, Bennett K, Boutilier K, et al. (2002) Systematic identification of protein complexes in Saccharomyces cerevisiae by mass spectrometry. Nature, 415:180-183
Houghten R A, Appel J R, Blondelle S E, Cuervo J H, Dooley C T, Pinilla C (1992) The use of synthetic peptide combinatorial libraries for the identification of bioactive peptides. Biotechniques 13(3):412-21.
Husi H, Ward M A, Choudhary J S, Blackstock W P, and Grant S G. (2000) Proteomic analysis of NMDA receptor-adhesion protein signaling complexes. Nature Neurosci. 3(7):661-9.;
Inglis S R, Stojkoski C, Branson K M, Cawthray J F, Fritz D, Wiadrowski E, Pye S M, Booker G W (2004) Identification and specificity studies of small molecule ligands for SH3 protein domains J. Med Chem. 47:5405-5417
Ingham R J, Colwill K, Howard C, Dettwiler S, Lim C S H, Yu J, Hersi K, Raajmakers J, Gish G, Mbamalu G, Taylor L, Yeung B, Vassilovski G, Amin, Chen F, Matskova L, Winberg G, Ernberg I, Linding R, O'Donnell P, Starosine A, Keller W, Metalnikov P, Stark C, Pawson T (2005) WW Domains provide a platform for the assembly of multiprotein networks Molecular and Cellular Biology 25(16):7092-7106
Iwabuchi, K., Li, B., Bartel, P. and Fields, S. (1993) Use of the two-hybrid system to identify the domain of p53 involved in oligomerization. Oncogene 8, 1693-1696.
Jin, Y., Yu, J., and Yu, Y. G. (2002). Identification of hNopp140 as a binding partner for doxorubicin with a phage display cloning method. Chem. Biol. 9, 157-162.
K Madura K, Dohmen RJ and Varshaysky A (1993) N-recognin/Ubc2 interactions in the N-end rule pathway. J. Biol. Chem.,268 (16), 12046-12054;
Kaida D, Motoyoshi H, Tashiro E, Nojima T, Hagiwara M, Ishidami K, Watanabe H, Kitahara T, Yoshida T, Nakajima H, Tani T, Horinouchi S, Yoshida M. (2007) Nature Chemical Biology 3(9):576-583
Kawamura, T., D. Liu, et al. (2003). “Anti-angiogenesis effects of borrelidin are mediated through distinct pathways: threonyl-tRNA synthetase and caspases are independently involved in suppression of proliferation and induction of apoptosis in endothelial cells.” J Antibiot (Tokyo) 56(8): 709-15.
Kotake Y, Sagane K, Owa T, Mimori-Kiyosue Y, Shimizu H, Uesugi M, Ishihama Y, Iwata M, Mizui Y. (2007) Nature Chemical Biology 3(9):570-575
Krawczak, M., Reiss, J., and Cooper, D. N. (1992). The mutational spectrum of single base pair substitutions in mRNA splice junctions of human genes: causes and consequences. Hum. Genet., 90, 41-54.
Lam K S, Salmon S E, Hersh E M, Hruby V J, Kazmierski W M, Knapp R J (1991). A new type of synthetic peptide library for identifying ligand-binding activity. Nature.;354(6348):82-84
Lam, (1997), Application of combinatorial library methods in cancer research and drug discovery. Anticancer Drug Des; 12:145-167
Lin K-T., Lu, R-M., Tarn, W-Y. (2004). The WW domain-containing protenis interact with the early spliceosome and participate in Pre-mRNA splicing in vivo. Mol. Cell Biol., 24, 9176-9185.
Matter, A,. (2001) Tumor angiogenesis as a therapeutic target. Drug Dis. Today 6:1005-1024.
McKenzie, M. K., Videlock, E. J., Splittgerber, U., and Austin, D. J. (2004). Simultaneous identification of multiple protein targets by using complementary-DNA phage display and a natural-product mimetic probe. Angew. Chem. Int. Ed. Engl., 43(31):4052-5.
Moss S J, Carletti I, Olano C, Sheridan R M, Ward M, Math V, Nur-E-Alam M, Brana A F, Zhang M Q, Leadlay P F, Mendez C, Salas J A, Wilkinson B (2006) Biosynthesis of the angiogenesis inhibitor borrelidin: directed biosynthesis of novel analogues Chem. Commun. 2006: 2341-2343
Nagamitsu, T., Takano, D., Fukuda, T., Otoguro, K., Kuwajima, I., Harigaya, Y. and Ōmura, S. (2004) Total synthesis of (−)-borrelidin. Org. Lett., 6, 1865-1867.
Olano C, Moss S J, Brana A F, Sheridan R M, Math V, Weston A J, Mendez C, Leadlay P F, Wilkinson B, Salas J A. (2004b) “Biosynthesis of the angiogenesis inhibitor borrelidin by Streptomyces parvulus Tu4055: insights into nitrile formation.” Mol. Microbiol., 52, 1745-1756.
Olano C, Wilkinson B, Moss S J, Brana A F, Mendez C, Leadlay P F, Salas J A. (2003), “Evidence from engineered gene fusions for the repeated use of a module in a modular polyketide synthase.” Chem. Commun, 2780-2782.
Olano C, Wilkinson B, Sanchez C, Moss S J, Sheridan R, Math V, Weston A J, Brana A F, Martin C J, Oliynyk M, Mendez C, Leadlay P F, Salas J A. (2004a) “Biosynthesis of the angiogenesis inhibitor borrelidin by Streptomyces parvulus Tu4055: cluster analysis and assignment of functions.” Chem. Biol., 11, 87-97.
Otoguru, K., Ui, H., Ishiyama, A., Kobayashi, M., Togashi, H., Takahashi, Y., Masuma, R., Tanaka, H., Tomado, H., Yamada, H., and Omura, S. (2003) In vitro and in vivo antimalarial activities of a non-glycosidic 18-membered macrolide antibiotic, borrelidin, against drug-resistant strains of Plasmodia. J. Antibiot. 56:727-729.
Paetz, W., and Nass, G. (1973) Biochemical and immunological characterization of threonyl-tRNA synthetase of two borrelidin-resistant mutants of Escherichia coli K12. Eur. J. Biochem. 35:331-337.
Perrin, R. M., Konopatskaya, O., Qiu, Y., Harper, S. J., Bates, D. O., and Churchill, A. J. (2005). Diabetic retinopathy is associated with a switch in splicing from anti- to pro-angiogenic isoforms of vascuar endothelial growth factor. Diabetoligia, 48, 2422-2427.
Rennel E S, Waine E, Guan H, Schuler Y, Leenders W, Woolard J, Sugiono M, Gillatt D, Kleinerman E S, Bates D O, Harper S J. (2008) British Journal of Cancer 98(7):1250-1257
Ruan B, Bovee M L, Sacher M, Stathopoulos C, Poralla K, Francklyn C S, Söll D. (2005) A unique hydrophobic cluster near the active site contributes to differences in borrelidin inhibition among threonyl-tRNA synthetases. J. Biol. Chem., 280, 571-577.

Sanford, J. R., and Caceres, J. F. (2004) Pre-mRNA splicing: life at the centre of the central dogma. J. Cell Sci., 117, 6261-6263.
Scott J K, Smith G P. (1990) Searching for peptide ligands with an epitope library. Science. 249(4967):386-390.
Singh, S. K., Gurusiddaiah, S., and Whalen, J. W. (1985) Treponemycin, a nitrile antibiotic active against Treponema hyodysenteriae. Antimicrob. Aqents Chemother. 27:239-245.
Usui, T., Watanabe, H., Nakayama, H., Tada, Y., Kanoh, N., Kondoh, M., Asao, T., Takio, K., Watanabe, H., Nishikawa, K., Kitahara, T., and Osada, H. (2004). The anticancer natural product pironetin selectively targets Lys352 of alpha-tubulin. Chem. Biol. 2004,11, 799-806.
Vong, B. G., Kim, S.-H., Abraham, S. and Theodorakis, E. A. (2004) Stereoselective total synthesis of (−)-borrelidin. Angew. Chem. Int. Ed., 43, 3947-3951.
Varey A H R, Rennel E S, Qiu Y, Bevan H S, Perrin R M, Raffy S, Dixon A R, Paraskeva C, Zaccheo O, Hassan A B, Harper S J, Bates D O. (2008) British Journal of Cancer 98(8):1366-1379
Wakabayashi, T., Kageyama, R., Naruse, N., Tsukahara, N., Funahashi, Y., Kitoh, K., and Watanabe, Y. (1997) Borrelidin is an angiogenesis inhibitor; disruption of angiogenic capilla vessels in a rat aorta matrix culture model. J. Antibiot. 50:671-676.
Wilkinson B, Gregory M A, Moss S J, Carletti I, Sheridan R M, Kaja A, Ward M, Olano C, Mendez C, Salas J A, Leadlay P F, vanGinkel R, Zhang M Q (2006). Separation of anti-angiogenic and cytotoxic activities of borrelidin by modification at the C17 side chain. Bioorganic and Medicinal Chemistry Letters 16:5814-5817

Woolard, J., Wang, W-Y., Bevan, H. S., Qui, Y., Morbidelli, L., Pritchard-Jones, R. O., Cui, T-G., Sugiono, M., Waine, E., Perrin, R., Foster, R., Digby-Bell, J., Shields, J. D., Whittles, C. E., Mushens, R. E., Gilliatt, D. A., Ziche, M., Harper, S. J., and Bates, D. O. (2004). VEGF₁₆₅b, an inhibitory vascular endothelial growth factor splice variant: mechanism of action, in vivo effect on angiogenesis and endogenous protein expression. Cancer Res., 64, 7822-7835.

Yamaoka, M., Sato, K., Kobayashi, M., Nishio, N., Ohkubo, M., Fujii, T., and Nakajima, H. (2005). FR177391, a new anti-hyperlipidemic agent from Serratia. iv. target identification and validation by chemical genetic approaches. J. Antibiot. 58, 654-662.
Zervos, A., Gyuris, J. & Brent, R., (1993) Mxl1, a protein that specifically interacts with max to bind myc-max recognition sites. Cell 72, 223-232.
Zuckermann R N, Martin E J, Spellmeyer D C, Stauber G B, Shoemaker K R, Kerr J M, Figliozzi G M, Goff D A, Siani M A, Simon R J, et al. 1994 Discovery of nanomolar ligands for 7-transmembrane G-protein-coupled receptors from a diverse N-(substituted)glycine peptoid library, J. Med. Chem. 37(17):2678-85.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A method of screening for anti-angiogenic agents which comprises identifying agents which interact with an FBP21 polypeptide or an FBP21 polypeptide fragment.

2. A method of screening for anti-angiogenic agents which comprises identifying agents which interact with one or both of the WW domains of an FBP21 polypeptide or an FBP21 polypeptide fragment.

3. A method according to claim 1, wherein the interaction comprises binding to the FBP21 polypeptide or FBP21 polypeptide fragment or one or both of the WW domains thereof

4. A method of treatment of conditions associated with angiogenesis which comprises administering to a patient in need thereof an anti-angiogenic agent which binds to FBP21 thereby to disrupt the interaction of FBP21 with its natural ligand.

5. A method according to claim 4 wherein the anti-angiogenic agent binds to one or both of the WW domains of FBP21.

6. (canceled)

7. (canceled)

8. An anti-angiogenic agent which binds to FBP21 thereby to disrupt the interaction of FBP21 with its natural ligand for use in the treatment of conditions associated with angiogenesis.

9. An anti-angiogenic agent according to claim 8 which binds to one or both of the WW domains of FBP21.

10. An agent according to claim 8 wherein the agent is an antibody.

11. An agent according to claim 10 wherein the antibody is a monoclonal antibody.

12. An agent according to claim 8 wherein the agent is a small molecule.

13. A method according to claim 1 which comprises the steps: (i) contacting a FBP21 polypeptide or a FBP21 polypeptide fragment with a candidate agent; and (ii) determining whether or not the candidate agent interacts with said polypeptide or polypeptide fragment.

14. A method according to claim 13 wherein the candidate agent is contacted with cells which produce FBP21 polypeptide in situ.

15. A method according to claim 13 wherein whether the determination of whether or not the candidate agent interacts with the FPB21 polypeptide or the FBP21 polypeptide fragment is made by reference to a control agent known to interact with the FPB21 polypeptide or the FBP21 polypeptide fragment.

16. A method according to claim 13 wherein the FBP21 polypeptide or the FBP21 polypeptide fragment or the candidate agent are labelled so as to enable detection of an interaction between the polypeptide or polypeptide fragment and the candidate agent.

17. A method according to claim 13 wherein the FBP21 polypeptide or the FBP21 polypeptide fragment and the candidate agent are contacted in a cell free system.

18. A method according to claim 17 wherein the FBP21 polypeptide or the FBP21 polypeptide fragment is immobilised on a solid support.

19. A method according to claim 13 wherein the determination of interaction of the candidate agent to the FBP21 polypeptide or the FBP21 polypeptide fragment is made in a competition assay employing a reference agent known to bind to the FBP21 polypeptide or the FBP21 polypeptide fragment.

20. A method of screening for anti-angiogenic agents which comprises identifying agents which modulate the activity or expression of FBP21 said method comprising the following steps: (i) contacting FBP21 expressing cells with a candidate agent; and (ii) determining whether or not the candidate agent modulates the activity or expression of FBP21 by monitoring a modulation in a detectable downstream response.

21. A method of screening for anti-angiogenic agents which comprises identifying agents which modulate the activity or expression of FBP21 said method comprising the following steps: (i) administering to an experimental animal a candidate agent; and (ii) determining whether or not the candidate agent modulates the activity or expression of FBP21 by monitoring a modulation in a detectable downstream response in the experimental animal.

22. A method of screening for anti-angiogenic agents which comprises identifying agents which modulate the activity or expression of FBP21 said method comprising the following steps: (i) administering to experimental animals respectively a candidate agent and a reference agent; and (ii) determining whether or not the candidate agent modulates the level of expression of FBP21 by monitoring the level of expression in the animals administered with the candidate agent by reference to the level of expression in the animals administered with the reference agent.

23. A method of treating diseases associated with angiogenesis by administering to a patient in need thereof an effective amount of an anti-antiogenic agent identified by means of a method of screening according to claim 1.
